(19) United States
(12) Patent Application Publication
(10) Pub. No.: US 2022/0218459 A1
(43) Pub. Date: Jul. 14, 2022

(54) SELF-CLEANING AORTIC BLOOD FILTER

(71) Applicant: VEOSOURCE SA, Lausanne (CH)

(72) Inventors: Vitali VERIN, Genève (CH); Olivier COQUOZ, Genève (CH); Alexandre ROMOSCANU, Genève (CH); Duncan KEEBLE, Newbury (GB); Kent STALKER, San Marcos, CA (US)

(21) Appl. No.: 17/611,645

(22) PCT Filed: May 18, 2020
(86) PCT No.: PCT/US2020/033468

§ 371 Date: Nov. 16, 2021

Related U.S. Application Data
(60) Provisional application No. 62/849,241, filed on May 17, 2019.

(51) Int. Cl. A61F 2/01; B05D 3/06

(52) U.S. Cl. CPC A61F 2/0105; B05D 3/06; A61F 2220/0008; A61F 2250/0098; A61F 2230/0013; A61F 2250/0023; A61F 2240/001; A61F 2210/0014; A61F 2002/018; A61F 2002/016; A61F 2230/0054

(57) ABSTRACT

A blood filter having a support structure and a filter structure. Various embodiments of the filtering assemblies may be utilized as a temporary implant or a permanent implant. The support structure, which serves as an anchor for the blood filter may define a larger porosity. The filter structure may define a smaller porosity, the smaller pores of which prevent emboli from passing therethrough. The support and filter structures may be made separately and assembled. In some embodiments, the filter structure actively slides on the support structure to accommodate the collapsing and deployment of the filter assembly. In some embodiments, a polymer film is selectively applied to the filter structure to provide a more uniform porosity over an outer bend radius of the filter structure. In some embodiments, the filter assembly is shaped to conform to the contours of the aortic arch while enabling self-cleaning of the filter structure.

### RELATED APPLICATIONS

This patent application claims the benefit of U.S. Provisional Application No. 62/849,241, filed May 17, 2019, the disclosure of which is incorporated by reference in its entirety.

### FIELD OF THE DISCLOSURE

The present disclosure is directed generally to implantable blood filter devices and more specifically to filter devices to protect the brain and other organs from emboli.

### BACKGROUND OF THE DISCLOSURE

Various conventional devices exist to contain or control the flow of thrombic material and atheroma debris. Examples of such devices include U.S. Pat. Nos. 6,712,834 and 6,866,680 to Yassour, et al., and U.S. Pat. No. 7,670,356 to Mazzocchi et al., which disclose blood filter devices designed to capture the debris material. A concern with capture filters is that they can foul to the extent that blockage of blood flow develops, with obvious consequences. Accordingly, these devices are typically unsuitable for long term or permanent implantation.

In another approach, U.S. Pat. No. 6,258,120 to McKenzie et al., U.S. Pat. No. 8,430,904 to Belson, U.S. Pat. No. 8,062,324 to Shimon et al., and U.S. Patent Application Publication No. 2009/0254172 to Grewe are directed to aortic diverters that divert emboli away from arteries. Diverter-type devices are limited to certain artery junction structures where flow diversion is a suitable substitute for filtering, and, in many instances, do not provide a positive barrier to emboli, either by design or because of the way they are mounted within the aorta. Furthermore, these devices can foul with debris build up over time, leaving no recourse for remedying the fouling, and so are not suitable for long term or permanent implantation. Also, diverter devices that are based on anchoring in the aorta require large diameter catheters for delivery. Other diverter-type devices include U.S. Pat. No. 8,460,335 to Carpenter, are held in place by the attendant deployment means, and thus suitable only for temporary service.

More recently, “self-cleaning” blood filters have been introduced, such as International Application No. WO 2015/173646 to Verin, et al., owned by the owner of the present application, and contents of which are hereby incorporated by reference herein in its entirety. Such self-cleaning blood filters can operate to provide a positive barrier that prevents emboli from entering the arteries from the aortic arch while enabling blood to flow through the structure, effectively keeping the structures clear of debris.

While the work of Verin et al. provides sound concepts for both temporary and permanent blood filters, putting these concepts into practice has raised special challenges. Self-cleaning blood filters that facilitate fabrication and deployment aspects would be welcomed.

### SUMMARY OF THE DISCLOSURE

Various embodiments of the disclosure disclose a blood filter having a support structure and a filter structure. These structures may have different porosities. For example, the support structure, which serves as an anchor for the blood filter may define a larger porosity, the larger pores of which promotes ample tissue growth therethrough that secures the device in place for permanent implant applications. The filter structure may define a smaller porosity, the smaller pores of which prevent emboli from passing therethrough. For fabrication and delivery purposes, the support and filter structures may be made separately and assembled. In some embodiments, the support structure presents a coarser mesh. In some embodiments, the support structure is an expanded metal structure, akin to a stent. In some embodiments, the filter structure presents a finer braided mesh that is overlaid on or otherwise attached to the support structure. In some embodiments, a polymer film is selectively applied to the filter structure and cooperates with the filter structure to provide a more uniform porosity over the filter structure.

Filtration of the blood includes deflection of emboli by the filter structure back into the aortic arch, as well as capture of emboli within the pores of the filter structure. In some embodiments, the filter structure is suspended away from the artery inlets. As such, the filter structure may be subject to cross flows during the cardiac cycle that may dislodge emboli from the filter structure, with the emboli being returned to the aortic arch and away from the inlets of the arteries. Accordingly, accumulation of emboli the filter structure is thereby reduced, such that the filter assembly is characterized as being “self-cleaning.”

Various embodiments of the filtering assemblies may be utilized as a temporary implant or a permanent implant. The elastic properties of the filtering assemblies enable minimally invasive delivery through blood vessels, and further enable collapsing the device for retrieval of temporary implantations.

Structurally, various embodiments of the disclosure disclose a filter assembly for filtering blood entering an artery from an aortic arch, comprising a filter structure and a support structure that cooperate to define an anchor leg and a filter leg. The anchor leg defines a distal opening and extends along a first central axis. The first central axis extends in an inferior direction from the distal opening toward an elbow portion of the filter leg, the filter leg being proximal to the anchor leg and defining and extending along a second central axis. The filter leg includes the elbow portion and an extension portion, the elbow portion extending from the anchor leg and separating the anchor leg from the extension portion. The support structure extends from the elbow portion into the extension portion.

In some embodiments, the filter structure is configured to slide on the support structure. The support structure may include at least one rail that extends into the extension portion, the filter structure being slidable on the rail. In some embodiments, the at least one rail may form a loop proximal to the filter structure. The loop may be configured as a snare portion for retrievability of the filter assembly, and may include a crimp attached to a proximal end of the loop, which may include a radiopaque material.

In some embodiments, the filter assembly defines an assembled length that extends from the first central axis at a distal opening of the anchor leg to an inside surface of the loop at the second central axis when the filter assembly is in a pre-implant configuration. The loop defines an inside length along the second central axis that extends from a proximal end of the filter structure to the inside surface of the loop when the filter assembly is in a pre-implant configuration. In some embodiments, a ratio of the assembled length to the inside length is in a range of 5 to 1 inclusive.

In some embodiments of the disclosure, the anchor leg includes a tail portion that extends distally beyond the assembled length, the tail portion being configured as a snare portion for retrievability of the filter assembly. The snare portion may include a hook structure. The tail portion may include a crimp attached to a distal end of the tail portion, the crimp including a radiopaque material.

In some embodiments of the disclosure, the filter structure and the support structure are interlaced. The filter structure may be one of a braided structure and a woven structure. In some embodiments, the filter structure and the support structure are tightly interlaced at the elbow portion to secure the filter structure to the support structure, while the filter structure and the support structure are loosely interlaced at the extension portion to enable the filter structure to slide over the support structure.

In some embodiments, the filter structure may include opposed lateral edges that are lateral to the second central axis, each of the opposed lateral edges including a hem structure that enables the filter structure to slide over the support structure. The extension portion of the filter structure defines an arcuate cross-section orthogonal to the second central axis that partially surrounds the second central axis, the arcuate cross section extending away from the central axis in the inferior direction. The arcuate cross-section may define one of a U-shape and a V-shape.

In some embodiments, a distal end of the filter structure is fastened to the anchor leg.

In some embodiments, a distal end of the filter structure defines closed neck. The distal end of the filter structure may be fastened to the anchor leg with crimps, and the crimps may include a radiopaque material suitable for visualization with an imaging system. In some embodiments, the closed neck wraps around anchor leg. In other embodiments, the distal end of the filter structure abuts against the anchor leg to define a diameter that is substantially the same as a diameter of the anchor leg. In some embodiments, the anchor support structure defines a first area porosity that is within a range of 60% to 98% inclusive. A mesh used to fabricate the filter structure may define a second area porosity that is within a range of 50% to 98% inclusive. A nominal pore size of the support structure may be greater than a nominal pore size of the filter leg. In some embodiments, the nominal pore size of the support structure is within a range of 0.5 to 8 millimeters inclusive; in some embodiments, within a range of 0.5 to 5 millimeters inclusive; in some embodiments, within a range of 0.5 to 3 millimeters inclusive. In some embodiments, the nominal pore size of the filter mesh of filter structure is within a range of 0.2 to 0.8 millimeter inclusive. In some embodiments, a ratio of the nominal pore size of the support structure to the nominal size of the filter mesh of filter structure is within a range of 2.5 to 55 inclusive; in some embodiments, within a range of 2.5 to 40 inclusive; in some embodiments, within a range of 2.5 to 25 inclusive.

When the filter assembly is in an implant configuration, a lateral projection of the first central axis and the second lateral axis may define a minimum projected angle, the minimum projected angle being within a range of 40 degrees to 80 degrees inclusive. In some embodiments, the minimum projected angle is within a range of 50 degrees to 70 degrees inclusive.

In various embodiments of the disclosure, a filter assembly for filtering blood entering an artery from an aortic arch is disclosed, comprising an anchor leg and a filter leg that extends from the anchor leg, the filter leg including an elbow portion, the anchor leg defining a first central axis that extends in a first direction from the anchor leg away from the elbow portion, and a support structure extending from the anchor leg along the filter leg, the support structure including a pair of support rails that extend beyond the elbow portion along the filter leg. A first support rail of the pair of support rails may define a first shape beyond the elbow portion, and a second support rail of the pair of support rails may define a second shape beyond the elbow portion. In some embodiments, the second shape extends further in the first direction than the first shape.

The first shape and the second shape may each arc toward a second direction, the second direction being opposite the first direction. The first shape and the second shape may each arc toward a first lateral direction, the first lateral direction being perpendicular to the first direction. In some embodiments, the anchor leg is configured to anchor the filter assembly in a brachiocephalic artery. The first shape and the second shape may each configured for continuous contact along a roof of an aortic arch, the continuous contact of the first shape being anterior to the continuous contact of the second shape In some embodiments, a filter structure is coupled to the pair of rails, the filter structure including a web portion that extends between the pair of rails. The filter structure may define an arcuate cross-section orthogonal to the second central axis, the arcuate cross section extending in a second direction that is opposite the first direction. In some embodiments, the cross-section defines one of a U-shape and a V-shape.

In various embodiments of the disclosure, filter assembly for filtering blood entering an artery from an aortic arch is disclosed, comprising a filter structure and a support structure that cooperate to define an anchor leg and a filter leg, the anchor leg defining a distal opening and extending along a first central axis, the first central axis extending in an inferior direction from the distal opening toward an elbow portion of the filter leg, the filter leg being proximal to the anchor leg and defining and extending along a second central axis, the filter leg including the elbow portion and an extension portion, the elbow portion extending from the anchor leg and separating the anchor leg from the extension portion, A perforated polymer coating may cover an outer contour of the elbow portion, and defines a plurality of perforations that pass through the perforated polymer coating. In some embodiments, the perforations of the plurality are sized within a range of 0.2 to 0.8 millimeter diameter inclusive. In some embodiments, the perforated polymer coating defines an area porosity that is within a range of 60% to 98% inclusive.

In various embodiments of the disclosure, a method of making this filter assembly comprises: coating the outer contour of the elbow portion with a polymer; and forming the plurality of perforations through the polymer. The polymer may be applied as a liquid and allowed to harden before the step of forming. The plurality of perforations may be formed by a laser cutting process. In some embodiments, the filter assembly is formed to shape over a mandrel and heat set to form the elbow portion prior to the step of coating.

In various embodiments of the disclosure, a method of collapsing a filter assembly for vascular delivery is disclosed, comprising: bending a filter assembly from an implant configuration to a pre-implant configuration; collapsing the filter assembly toward a central axis of the pre-implant configuration; and sliding at least a portion of a filter structure of the filter assembly along a support structure of the filter assembly during the step of collapsing to elongate the filter structure along the central axis of the pre-implant configuration. The filter structure may slide along a rail of the support structure during the step of sliding. The filter structure may include a hem that slides along the rail of the support structure during the step of sliding. The

filter assembly in the steps of bending and collapsing may be made of super-elastic material, such as a nickel titanium alloy and a cobalt-chromium-nickel-molybdenum-iron alloy.

In various embodiments of the disclosure, a method of forming rails on a support structure of a filter assembly is disclosed, comprising: forming a plurality of pre-expansion pores at a first end portion of a tube to define a pre-expansion anchor portion; cutting at least one segment proximal to the pre-expansion anchor portion to form at least one rail extending proximal to the pre-expansion anchor portion; and expanding the tube to define an expanded anchor portion. In some embodiments, a ratio of a length that the rail extends from the expanded anchor portion to a length of the expanded anchor portion is in range of 0.2 to 1.5 inclusive. A taper may be formed at a distal end of the pre-expansion anchor portion. In some embodiments, the method includes coupling a filter structure to the at least one rail portion, the filter structure including a proximal end, and closing the at least one rail portion to form a loop with the proximal end of the filter portion to support the filter structure. During the step of coupling, the filter structure may include capturing the at least one rail portion within a hem structure of the filter structure. In some embodiments, the at least one rail portion in the step of cutting at least one segment is two rail portions, wherein the step of closing may include joining proximal ends of the two rail portions together. The tube in the step of forming the plurality of pre-expansion pores may be a circular tube. In some embodiments, the steps of cutting are performed with a laser.

In various embodiments of the disclosure, a method of forming a filter assembly for a blood filter is disclosed, comprising: forming a tubular sleeve structure defining a substantially linear central axis, the tubular sleeve structure defining a wall porosity; partially severing the tubular sleeve structure to form severed edges that are bridged by a hinge portion, the hinge portion extending along one side of the tubular sleeve structure; rolling or folding the severed edges back along an inside of the tubular sleeve structure to define opposed mitered edges, the opposed mitered edges defining a miter angle when the tubular sleeve defines the substantially linear central axis; and closing the miter angle about the hinge portion to define an elbow shape. In some embodiments, the tubular sleeve is mounted on a mandrel to close the miter angle, and may be heat setting the tubular sleeve on the mandrel. The method may include the step of sliding the sleeve structure over a support structure to close the miter angle.

In various embodiments of the disclosure, a filter assembly for filtering blood flowing into an artery is disclosed, comprising a tubular support structure that defines a first open end and a second open end that is opposed to the first open end, the tubular support structure having a tubular wall that defines a wall area porosity, the tubular support structure being curved to define a first leg portion and a second leg portion, the second leg portion including an elbow portion that extends from the first leg portion, the first leg portion defining the first open end and a first central axis, the second leg portion defining the second open end and a second central axis, the first central axis and the second central axis defining a minimum projected angle of the first central axis and the second central axis that is less than 180 degrees. A filter structure may define a filter area porosity and may be coupled to the second leg portion of the tubular support structure, In some embodiments, the filter assembly defines an inside portion that faces inward and an opposed outside portion that faces outward. The filter structure and the tubular support structure may define a combined area porosity that is less than the wall area porosity. In some embodiments, the filter structure is arranged so that at least part of the outside portion of the filter assembly defines the combined area porosity at the elbow portion and the second leg portion, and at least part of the inside portion defines the wall area porosity at the second leg portion. In some embodiments, the minimum projected angle is an obtuse angle; in others, the minimum projected angle is an acute angle. In some embodiments, the minimum projected angle is in a range of 40 degrees to 80 degrees inclusive; in some embodiments, the minimum projected angle is in a range of 50 degrees to 70 degrees inclusive.

In some embodiments, the filter structure is disposed on an interior of the tubular support structure; in others, the filter structure is disposed on an exterior of the tubular support structure. The filter structure may be attached to the tubular support structure with at least one of a threaded wire, a plurality of stitches, and a plurality of point-wise tack welds. In some embodiments, the tubular support structure includes one of a braided structure and a woven structure, the tubular support structure including a plurality of pores defined therethrough.

The tubular support structure may be of a coarse wire mesh, wherein the coarse wire mesh includes wire having a diameter in a range of 100 micrometers to 300 micrometers inclusive and defining pore sizes in a range of three millimeters to five millimeters inclusive, the coarse wire mesh being one of a braided structure and a woven structure. The coarse wire mesh may be formed from a single continuous wire. In some embodiments, wire is composed of a material that includes one of a cobalt-chromium-nickel-molybdenum-iron alloy and a nickel-titanium alloy. The material may be one of NITINOL and an alloy specified by ASTM F 1058 or ISO 5832-7.

In some embodiments, the filter structure is a two-dimensional structure that conforms to a shape of the tubular support structure when coupled to the tubular support structure. The filter structure may be one of a braided structure and a woven structure that is integrated with the elbow portion and the second leg portion. In some embodiments, the filter structure is a fine wire mesh, wherein the fine wire mesh is braided or woven with wire having a diameter in a range of 30 micrometers to 100 micrometers inclusive. The fine wire mesh may define a plurality of non-circular pores, each having a nominal major dimension in a range of 200 micrometers to 800 micrometers inclusive and may be woven or braided from a single wire.

In some embodiments, the wire is of a super elastic material, such as NITINOL.

In various embodiments of the disclosure, a method of manufacturing a filter assembly is disclosed, comprising: forming the tubular support structure about a substantially linear axis; fitting the tubular support structure over a curved mandrel to define the minimum projected angle; heat treating the tubular support structure on the mandrel; and coupling the filter structure to the elbow portion and the leg portion. The filter structure may be a tubular sleeve structure defining a plurality of apertures formed on a first side thereof, the plurality of apertures being arranged so that the inside portion of the filter assembly defines the wall area porosity of the tubular support structure through the plurality of apertures. In some embodiments, one or more of the plurality of apertures is arranged on the first side for substantial alignment with ostia of arteries that branch from an aortic arch when the filter assembly is implanted in the aortic arch.

In various embodiments of the disclosure, a method of manufacturing a filter assembly, includes: forming the tubular sleeve structure of the filter structure about a substantially linear axis; fitting the tubular sleeve structure over a mandrel, the mandrel including a plurality of apertures on one side that pass through a wall of the mandrel into a hollow defined by the mandrel; heat treating the tubular support structure on the mandrel; forming a plurality apertures in the tubular sleeve structure that pass through the plurality of apertures of the mandrel; and coupling the filter structure to the elbow portion and the leg portion of the tubular support structure. The step of coupling the filter structure to the elbow portion and the second leg portion of the tubular support structure may include arranging the filter structure on an exterior of the tubular support structure, and the step of forming the tubular support structure may include one of a weaving or a braiding process.

In various embodiments of the disclosure, a filter assembly is disclosed, comprising a tubular support structure that defines a first open end and a second open end that is opposed to the first open end, the tubular support structure having a tubular wall that defines a wall area porosity, the tubular support structure being curved to define a first leg portion and a second leg portion separated by an elbow portion, the first leg portion defining the first open end and a first central axis, the second leg portion defining the second open end and a second central axis, the first central axis and the second central axis intersecting to define an apex angle that is less than 180 degrees, the apex angle defining a central plane of the tubular support structure. A filter structure may define a filter area porosity and being coupled to the elbow portion and the second leg portion of the tubular support structure. In some embodiments: the filter assembly defines an inside portion that faces toward the apex angle and an opposed outside portion that faces away from the apex angle; the filter structure and the tubular support structure define a combined area porosity that is less than the wall area porosity; and the filter structure is arranged so that at least part of the outside portion of the filter assembly defines the combined area porosity at the elbow portion and the second leg portion, and at least part of the inside portion defines the wall area porosity at the second leg portion.

### BRIEF DESCRIPTION OF THE DRAWINGS

FIG. 1 is a frontal cutaway view of a human heart with a filter device implanted in an embodiment of the disclosure;

FIG. 2 is an exploded perspective view of a filter assembly having a support structure and a filter structure according to an embodiment of the disclosure;

FIG. 3 is an assembled perspective view of the filter device of FIG. 2 according to an embodiment of the disclosure;

FIG. 4 is a partial plan view of a porous construction for the filter structure of FIGS. 2 and 3 according to an embodiment of the disclosure;

FIG. 5 is a perspective view of a support structure according to an embodiment of the disclosure;

FIG. 6 is a plan view of a flat filter portion mounted on a loom frame according to an embodiment of the disclosure;

FIG. 7 is a schematic view of a filter mesh according to an embodiment of the disclosure;

FIG. 8 is an enlarged, partial view of the schematic view of the filter mesh of FIG. 7 according to an embodiment of the disclosure;

FIGS. 9 and 10 are perspective views of a filter structure and a corresponding mandrel for formation thereof according to an embodiment of the disclosure;

FIG. 11 is a tubular filter structure prior to shaping according to an embodiment of the disclosure;

FIG. 12 is the tubular filter structure of FIG. 11 after shaping with a perforated polymer overcoat according to an embodiment of the disclosure;

FIG. 13 is a partial, enlarged sectional view of an elbow portion of the tubular filter structure of claim 12 prior to overcoating according to an embodiment of the disclosure;

FIG. 14 is the sectional view of FIG. 13 after application of a liquid polymer coating according to an embodiment of the disclosure;

FIG. 15 is the sectional view of FIG. 14 after perforation of the polymer overcoat according to an embodiment of the disclosure;

FIG. 16 is a perspective view of a mitered tubular filter structure prior to shaping according to an embodiment of the disclosure;

FIG. 17 is a perspective view of the mitered tubular filter structure of FIG. 16 after shaping according to an embodiment of the disclosure;

FIG. 18 is a perspective view of a filter assembly with a filter structure interlaced with a support structure in a pre-implant configuration according to an embodiment of the disclosure;

FIG. 19 is a perspective view of the filter assembly of FIG. 18 in an implant configuration according to an embodiment of the disclosure;

FIG. 20 is a perspective view of a filter assembly in an implant configuration and having a filter structure with hemmed lateral edges for sliding on a support structure according to an embodiment of the disclosure;

FIG. 21 is a planar projection of a pre-expanded tube of the support structure of the filter assembly of FIG. 20 according to an embodiment of the disclosure;

FIG. 22 is an enlarged, partial view of the planar projection of FIG. 21 according to an embodiment of the disclosure;

FIG. 23 is a planar projection of the tube of FIG. 21 in an expanded configuration according to an embodiment of the disclosure;

FIG. 24 is an enlarged, partial view of the support structure of FIG. 23 according to an embodiment of the disclosure;

FIG. 25 is a partial perspective view the support structure of the filter assembly of FIG. 20 according to an embodiment of the disclosure;

FIG. 26 is a partial elevational view of the support structure of FIG. 25 according to an embodiment of the disclosure;

FIGS. 27 through 29 are perspective views of a filter structure of the filter assembly of FIG. 20 according to an embodiment of the disclosure;

FIG. 30 is a cutaway perspective view of a filter assembly implanted in an aortic arch according to an embodiment of the disclosure; and

FIGS. 31 through 33 is a three-way orthographic projection of a shape of the filter assembly of FIG. 30 according to an embodiment of the disclosure.

### DETAILED DESCRIPTION

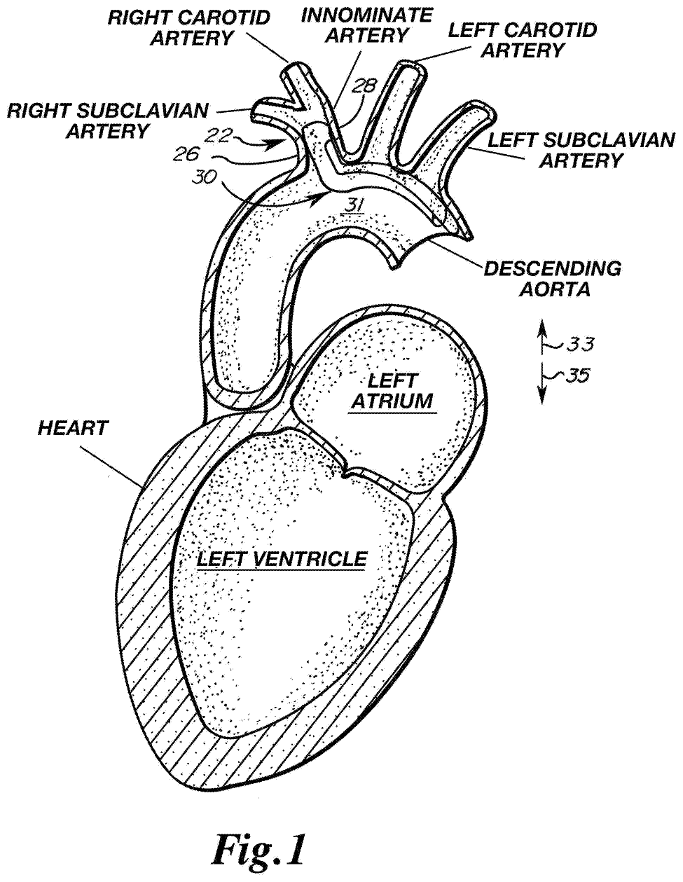

Referring to FIG. 1, an implantable filter device or assembly 30 is depicted in an implanted configuration 22 in an artery 26 of an aortic arch 31 of a heart. The filter assembly 30 is depicted as being implanted in the ostium (take-off) of an artery 26, and more specifically into a branch 28 of the aortic arch 31 to filter the blood flowing into the artery 26 from the aortic arch 31. The FIG. 1 depiction also presents, without limitation, various candidate arteries for implantation of the filter assembly 30, including the innominate artery (and attendant right carotid and right subclavian arteries), the left carotid artery, and the left subclavian artery. The FIG. 1 depiction also identifies a superior direction 33 and an inferior direction 35 of the anatomy. Herein, “superior” and “inferior” refer to standard anatomical terms for the orientation of the filter assembly 30 when implanted in the artery 26 of the aortic arch 31.

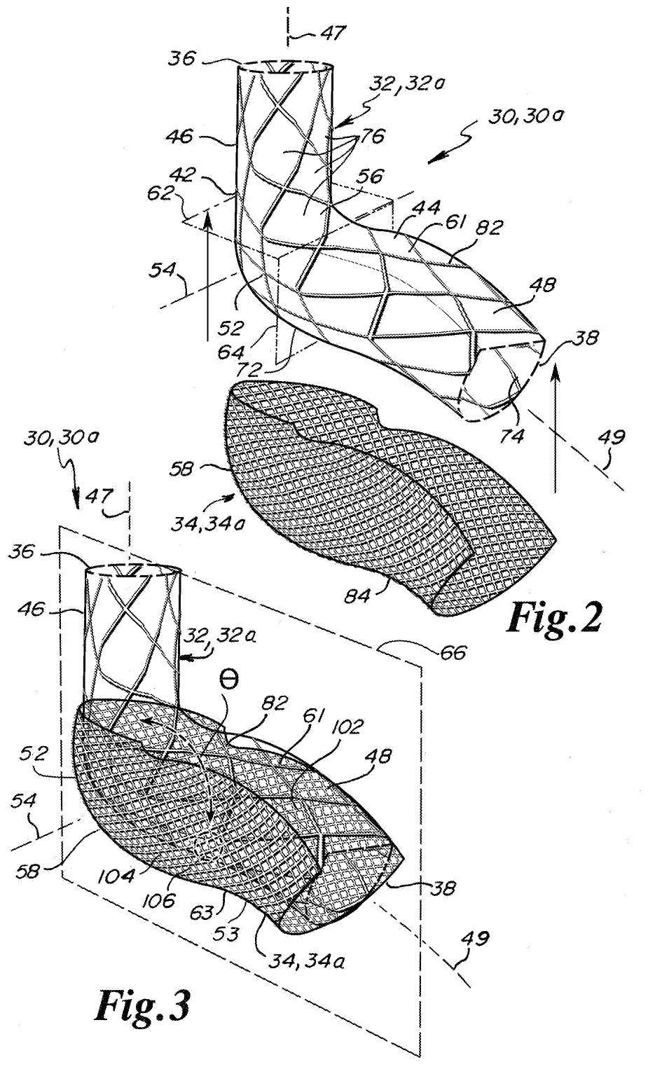

Referring to FIGS. 2 through 4, the filter assembly 30 is depicted for filtering blood flow into an artery according to embodiments of the disclosure. The filter assembly 30 includes a support structure 32 and a filter portion or structure 34. The filter assembly 30 includes an anchor leg portion 46 and a filter leg portion 48, the filter leg portion 48 including an elbow portion 52 that depends from the anchor leg portion 46 and an extension portion 53 that extends from said elbow portion 52. The anchor leg portion 46 defines and extends along a first central axis 47, and the filter leg portion 48 defines and extends along a second central axis 49. The elbow portion 52 is arcuate about a lateral axis 54 to define an inner contour 56 and an outer contour 58.

The anchor leg portion 46 defines a first opening 36, the first central axis 47 being concentric with a center of the first opening 36. The filter leg portion 48 defines a superior side 61 and an inferior side 63 of the filter assembly 30. The first central axis 47 and the second central axis 49 project a minimum projected angle θ onto a lateral projection plane 66, the minimum projected angle θ being defined about the lateral axis 54 and being less than 180 degrees.

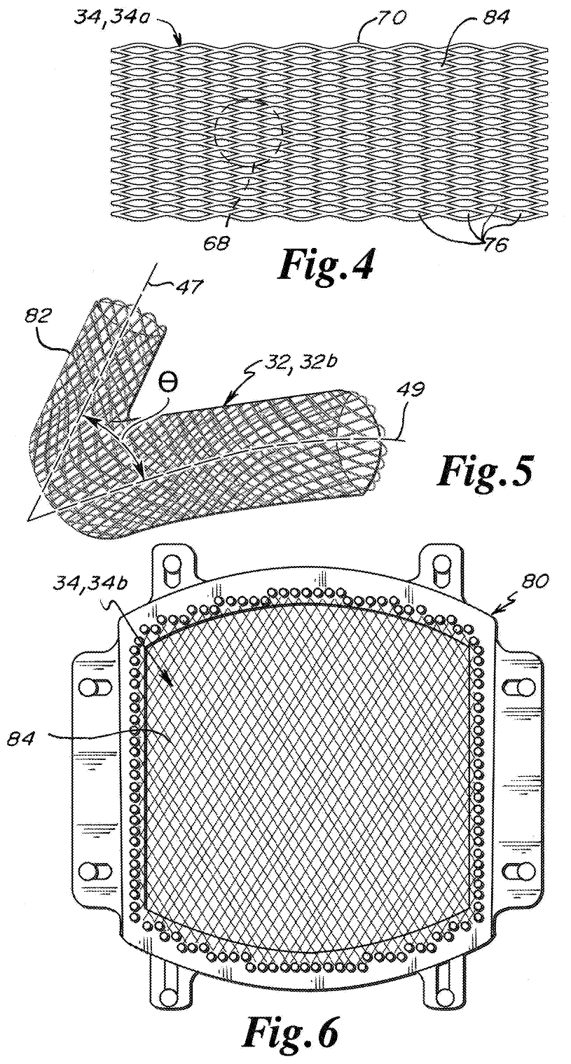

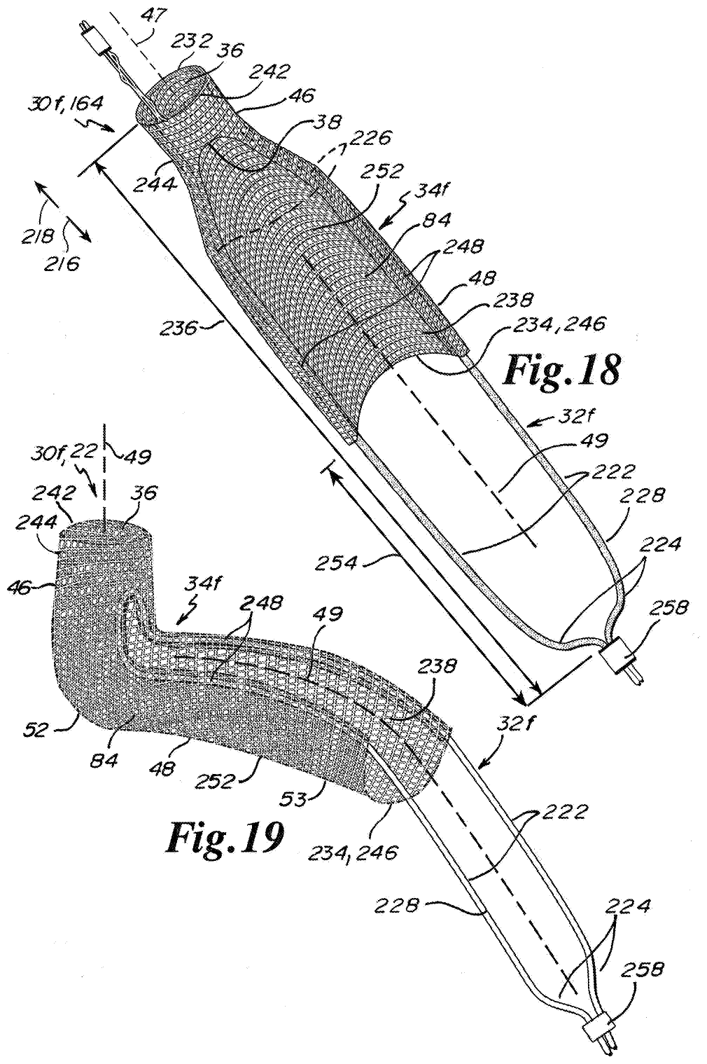

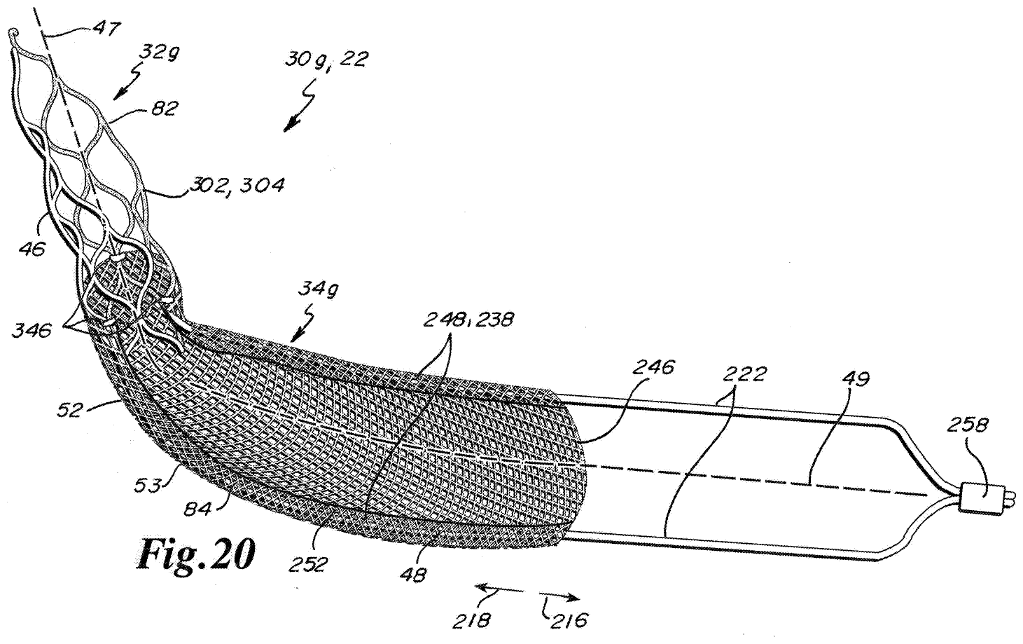

For filter leg portions 48 having components with cross-sections normal to and surrounding the second central axis 49 along the extension portion 53, the second central axis 49 is defined as concentric with the filter leg portion 48. Examples of such components include the support structures 32a and 32b of FIGS. 3 and 5, respectively, and the filter elements 34c, 34d, and 34e of FIGS. 9, 12, and 17, respectively. For filter leg portions 48 that do not surround the second central axis 49 along the extension portion 53 but instead have components with cross-sections normal to the second central axis 49 that only partially surround the second central axis 49, the central axis 49 is defined as being centered between opposed lateral edges of the filter leg portion 48. Examples include filter structures 34f and 34g of FIGS. 19 and 20, respectively, each of which define opposed lateral edge portions 248.

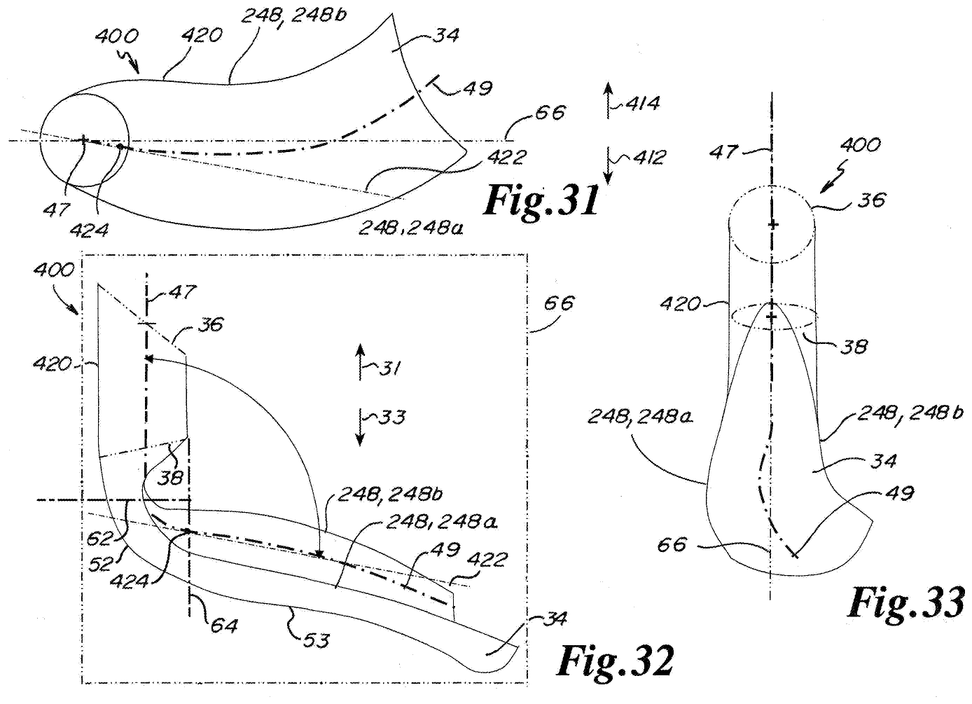

The elbow portion 52 constitutes a segment of the filter assembly 30 that is bounded distally by boundary plane 62 and proximally by boundary plane 64. Boundary plane 62 is normal to the first central axis 47 and intersects the first central axis 47 where the filter assembly 30 exits the ostium of the anchoring artery when properly implanted. Boundary plane 64 intersects the second central axis 49, is orthogonal to the lateral projection plane 66, and is tangent to an edge of the anchor leg portion 46. The depiction of FIG. 2 presents boundary planes 62 and 64 for a filter assembly 30 having a tubular filter leg portion 48. Boundary planes 62 and 64 are also depicted at FIG. 32 for a filter assembly 30 having a channel-shaped extension portion 53 of the filter leg portion 48.

In some embodiments, the anchor leg portion 46 of the filter assembly 30 is dimensioned for anchoring within an innominate (brachiocephalic) artery. The filter leg portion 48 may extend proximally to a length long enough to cover the ostium of the left common carotid artery when implanted in an innominate artery of the aortic arch 31 of the heart. In some embodiments, the filter leg portion 48 is long enough to cover the ostia of both the left common carotid artery and the left subclavian artery when implanted in the innominate artery.

Functionally, the support structure 32 supports the filter structure 34 in a preferred orientation that filters blood entering one, some, or all of the inlets of the innominate artery, the left common carotid artery, and the left subclavian arteries at the aortic arch. During implantation, the anchor leg portion 46 of the support structure 32 is implanted within an anchoring artery (e.g., the brachiocephalic artery). The filter leg portion 48 may be oriented to extend over the inlets of arteries proximate the anchoring artery (e.g., the left common carotid artery and the left subclavian arteries). The anchor leg portion 46 is inserted into the anchoring artery 26, contacting the walls of the anchoring artery 26 and bringing the filter leg portion 48 into contact with a superior surface of the aortic arch 31.

This disclosure presents several embodiments of filter assemblies 30, all of which have in common the support structure 32 and the filter structure 34, albeit in configurations that differ from those presented in FIGS. 2 and 3. To distinguish the various embodiments, the filter assemblies, support structures, and filter structures are herein referred to generically or collectively by the reference characters 30, 32, and 34, respectively, and specifically or individually by these reference characters followed by a letter suffix (e.g., filter assembly 30a having the support structure 32a and filter structure 34a of FIGS. 2 and 3).

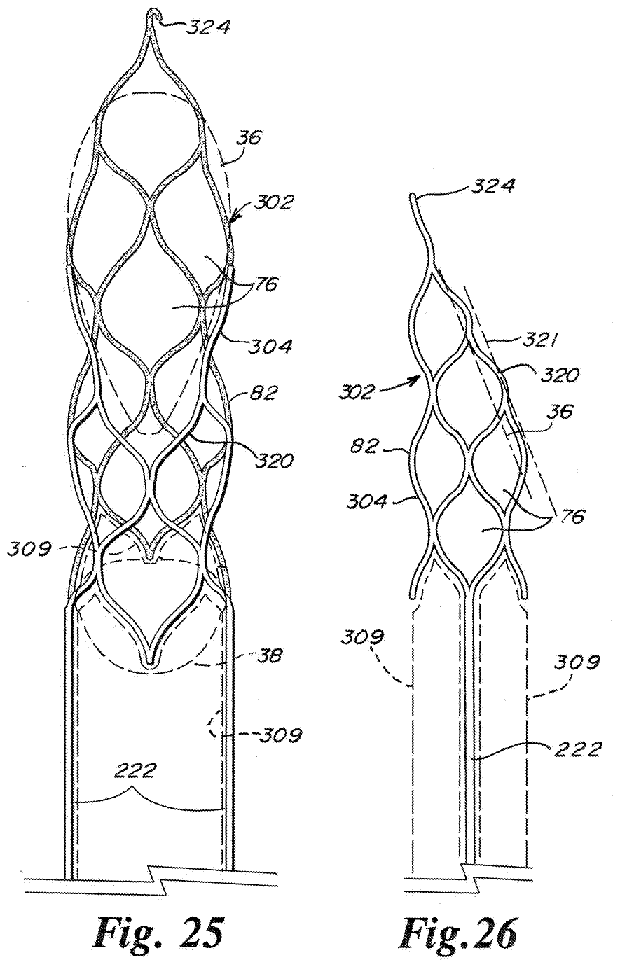

For the filter assembly 30a, the support structure 32a includes a tubular wall 42 that defines an area porosity 44. The filter assembly 30a also defines a second opening 38. For the filter assembly 30a, the second opening 38 is defined by the filter leg portion 48, and both of the openings 36 and 38 are be defined by the support structure 34a. In some embodiments, the second opening 38 is defined by the anchor leg portion 46 (FIG. 25) or within the elbow portion 52, and one or both of the openings 36 and 38 may be defined by the filter structure 34 (FIG. 18).

The filter assembly 30a defines an inside portion 102 that faces inward and an opposed outside portion 104 that faces outward. The filter structure 34a and the support structure 32a define a combined area porosity 106 that is less than the area porosity 44 of the support structure 32a. In some embodiments, the filter structure 34a is arranged so that at least part of the outside portion 104 of the filter assembly 30a defines the combined area porosity 106 at the filter leg portion 48, and at least part of the inside portion 102 defines the area porosity 44 at the filter leg portion 48. Herein, an “area porosity” is defined by a ratio of the normal projected area of the voids of a porous material to the total normal area of the porous material.

In some embodiments, the mesh 84 from which the filter structure 34 is fabricated defines a porosity in the range of 50% to 98% inclusive; in some embodiments, in a range from 60% and 95% inclusive; in some embodiments, in a range from 70% and 95% inclusive; in some embodiments, in a range from 75% and 90% inclusive. In some embodiments, the support structure defines a porosity in the range of 60% to 98% inclusive; in some embodiments, in a range from 70% and 95% inclusive; in some embodiments, in a range from 75% and 95% inclusive; in some embodiments, in a range from 80% and 95% inclusive.

In some embodiments, a coarse wire mesh 82 is exposed on the inside portion 102 of the filter assembly 30a that contacts the superior surface of the aortic arch surrounding the inlets of the arteries. The pores of the coarse wire mesh 82, being larger than the pores of the filter structure 34, enables filtered blood to flow into artery inlets without further obstruction.

For permanent implants, the larger pores 76 of the coarse wire mesh 82 of the support structure 32 also facilitates securing the filter assembly 30. Typically, after about 4 weeks' time, tissue on the anchoring artery grow into the larger pores 76 of the coarse wire mesh 82 of the anchor leg portion 46, and also enables the contacted tissue on the aortic arch to grow into the pores of the filter leg portion 48. The growth of the tissue into the larger pores 76 of the support structure 32 secures the filter assembly 30 in the preferred orientation.

In some embodiments, the filter structure 34a is fabricated from an expanded sheet 70 (e.g., expanded metal; FIG. 4) that defines a filter area porosity 68 and is coupled to filter leg portion 48. For the filter assembly 30a, the filter leg portion 48 includes the support structure 32a and the filter structure 34a, with the filter structure 34a disposed on an exterior 72 of the support structure 32a. Alternatively, the filter structure 34a may be disposed on an interior 74 of the support structure 32a. One or both of the support structure 32, 32a and the filter structure 34, 34a defines a plurality of pores 76.

In some embodiments, a method of manufacturing the filter assembly 30a includes weaving or braiding the support structure 32a about a substantially linear axis. Herein, “weaving” is a process that creates fixed cross-over points between crossing wires or filaments, whereas “braiding” is a process where the crossing wires or filaments are not fixed (i.e., crossing wires or filaments can slide with respect to each other). The support structure 32a is fitted over a curved mandrel (not depicted) to define the projected angle θ, and may be heat set on the mandrel to thermally set the curved shape of the support structure 32a. The filter structure 34a is then coupled to the filter leg portion 48 of the support structure 32a. The filter structure 34a may be attached to the support structure 32a with a threaded wire, a plurality of stitches, a plurality of point-wise tack welds, or a combination of such techniques. In some embodiments, the filter structure 34a is braided directly onto the filter leg filter leg portion 48 of the support structure 32a.

Referring to FIGS. 5 through 8, a support structure 32b and a filter structure 34b are depicted according to an embodiment of the disclosure. The support structure 32b may be formed to shape as described for the support structure 32a. The support structure 32b defines a coarse mesh 82, and the filter structure 34a defines a fine mesh 84. Herein, “coarse” and “fine” in relation to the meshes 82 and 84 are terms that are relative to each other. That is, the coarse wire mesh 82 is characterized as defining nominally larger pore sizes than the fine mesh 84, for example with a braiding from larger diameter wire. For the filter structure 34b, the mesh 84 is braided or woven, the filter structure 34b being depicted on a fabrication fixture 80.

In some embodiments, the coarse mesh 82 is braided or woven with wire having a diameter in a range of 100 micrometers to 300 micrometers inclusive, with nominal pore sizes in a range of 0.5 millimeters to five millimeters inclusive. In some embodiments, the nominal pore sizes are in a range of two millimeters to seven millimeters inclusive. (Herein, a range that is said to be “inclusive” includes the endpoint values of the range as well as all values therebetween.) In some embodiments, a ratio of the nominal pore size of the support structure 32 to the nominal size of the filter mesh 84 of filter structure 34 is within a range of 2.5 to 55 inclusive; in some embodiments, the ratio is within a range of 2.5 to 40 inclusive; in some embodiments, the ratio is within a range of 2.5 to 25. In some embodiments, the coarse mesh 82 is a woven mesh and may be braided from a single wire. Likewise, in some embodiments, the fine mesh 84 is a woven mesh that may be braided from a single wire.

Herein, a “pore” is a void bounded by a structural component or components, for example the wires of a woven mesh (e.g., pores 76 of meshes 82 and 84 of FIGS. 2 and 8). In some embodiments, the pores 76 result from a process of braiding or weaving metal wires or a polymer filaments (e.g., meshes 82, 84), for example by weaving or knitting. In some embodiments, the pores 76 result from an expanded metal process. In some embodiments, the pores 76 are formed on a structure, for example, a metal or polymer (e.g., the expanded sheet 70 of FIG. 4). Formation may be performed with laser cutting or other manufacturing techniques available to the artisan.

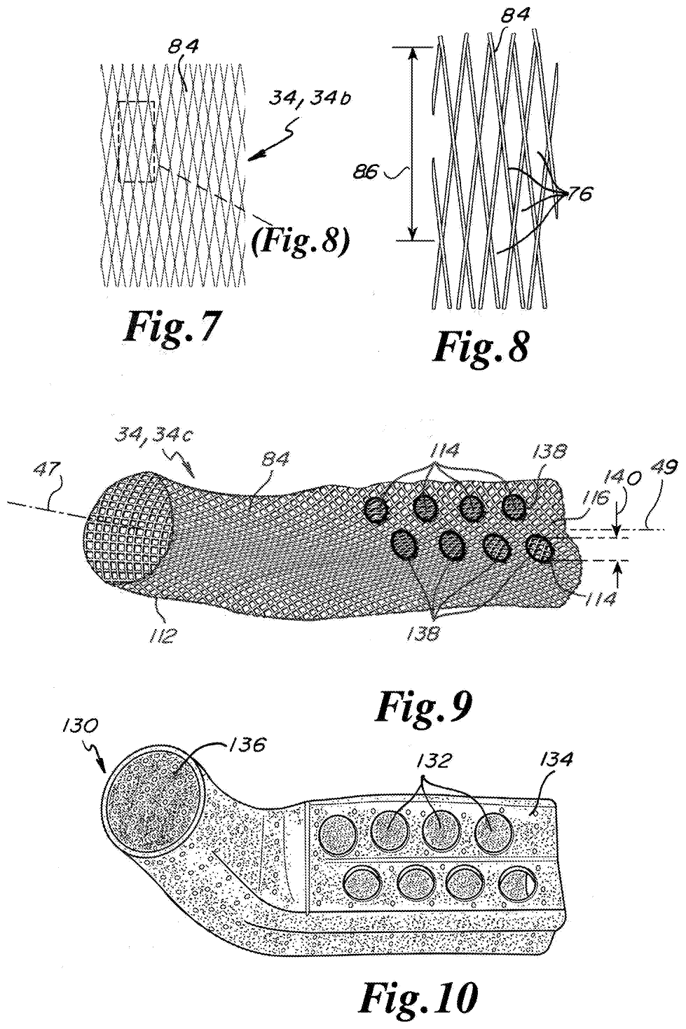

“Pore size” is defined as the diameter of a largest circle 110 that will fit within the inner dimensions of the pore 76. An illustration is depicted at FIG. 24. In some embodiments, the nominal pore sizes of a coarse mesh 82 are in a range of three millimeters to five millimeters inclusive. The fine mesh 84 may be braided with wire having a diameter in a range of 30 micrometers to 150 micrometers inclusive, with nominal pore sizes in a range 200 micrometers to 800 micrometers inclusive. In some embodiments, the filter structure 34 is a two-dimensional structure (e.g., FIG. 7) that conforms to a shape of the support structure 32 when coupled to support structure 32. In some embodiments, the pores 76 are elongate (e.g., neither circular nor square), defining a major dimension 86 (FIG. 8).

Examples of suitable metallic materials for the various filter assemblies 30 include so-called “super elastic” alloys such as certain nickel-titanium alloys (e.g., NITINOL), which allows up to 8% elastic deformation. Other examples of sufficiently elastic alloys include cobalt-chromium-nickel-molybdenum-iron (CoCrNi) alloys specified by ASTM F 1058 and ISO 5832-7, such as ELGILOY®, PHYNOX®, CONICHROME®, and FWM® 1058. Such CoCrNi alloys, though not “super elastic”, possesses sufficient elasticity by virtue of a high yield stress.

Referring to FIG. 9, a filter structure 34c having a plurality of apertures 114 on a superior side 116 is depicted according to an embodiment of the disclosure. The filter structure 34c may include various components and attributes as the filter assembly 30a and filter structures 34a and 34b, which are indicated with same-numbered reference characters. For the filter structure 34c, a tubular sleeve structure 112 is formed to shape and heat set. The plurality of apertures 114 are formed on the superior side 116 and may be sized greater than the pores 76 of the support structure 32 so that the wall area porosity 44 of the support structure 32 (FIG. 3) defines the porosity of the filter assembly 30 through the plurality of apertures 114. In some embodiments, one or more of the plurality of apertures 114 on the superior side 116 of the extension portion 35 of the filter structure 34c is arranged for substantial alignment with ostia of arteries that branch from the aortic arch 31.

Herein, an “aperture” is a hole formed through a material, for example by cutting, punching, flaring, or by braiding about a mandrel. Accordingly, the apertures 114 are distinguishable from the pores 76 of the meshes 82 or 84. That is, when formed on the mesh 82 or 84, an aperture 114 refers to a through-hole defined by or through the mesh 82, 84 that is larger than the pores of the mesh 82, 84.

Referring to FIG. 10, a mandrel 130 for shaping the filter structure 34c and forming the plurality of apertures 114 therein is depicted according to an embodiment of the disclosure. In some embodiments, fabrication of the filter structure 34c includes braiding the tubular sleeve structure 112 of the filter structure 34c about a substantially linear axis and fitting the tubular sleeve structure 112 over a mandrel 130, the mandrel 130 including a plurality of apertures 132 that pass through a wall superior 134 of the mandrel 130 into a hollow 136 defined the mandrel 130. The tubular support structure 112 may be heat treated on the mandrel 130 to thermally set the shape of the tubular support structure 112.

In some embodiments, the plurality of apertures 114 of the tubular sleeve structure 112 are formed using the plurality of apertures 132 of the mandrel 130 as a guide, for example by passing a punch or a flare tool through the tubular sleeve structure 112 and into the apertures 132 of the mandrel 130. In some embodiments, excess mesh material from the formation of the apertures 132 is rolled or folded back into the tubular sleeve structure 112 to form rims 138 about the apertures 132. In some embodiments, the apertures 132 define an aperture diameter 140 that is within a range of three millimeters to eight millimeters inclusive.

The filter structure 34 is then coupled to the support structure 32, for example the support structures 32a or 32b. Other support structures 32 disclosed throughout this disclosure may also be utilized. In some embodiments, the step of coupling the filter structure 34 to the support structure 32 includes arranging the filter structure 34c on the exterior 72 (FIG. 2).

Functionally, the plurality of apertures 114 can facilitate tissue growth into the filter leg portion 48, which is desirable for permanent implants. Those apertures 114 which align with the ostia prevent reduction of blood flow into the arteries that would otherwise be caused by the presence of the mesh 35 over the ostia.

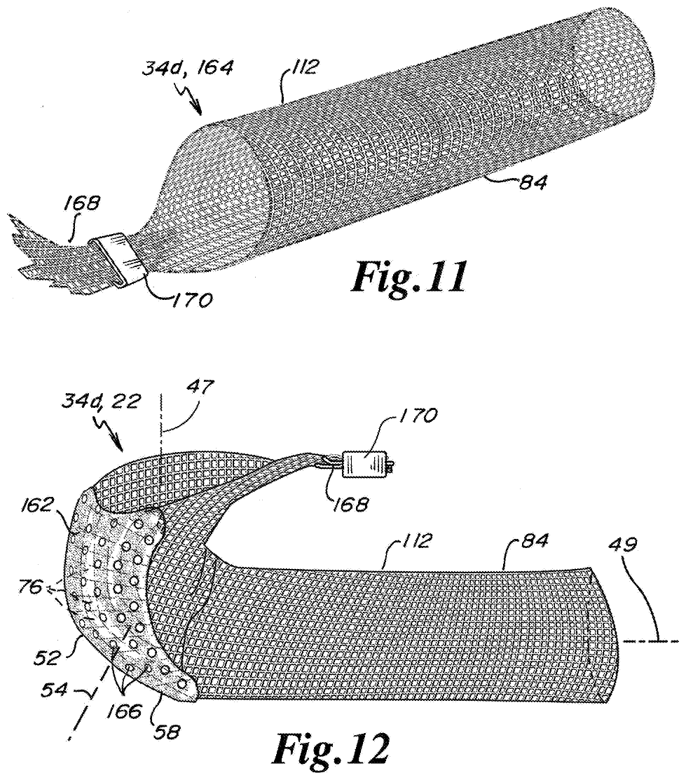

Referring to FIGS. 11 through 15 a filter structure 34d with a perforated polymer overcoat 162 is depicted according to an embodiment of the disclosure. The filter structure 34d is depicted in a pre-implant configuration 164 (FIG. 11) and the implanted configuration 22 (FIG. 12). The filter structure 34d may include some components and attributes as the filter structure 34c, some of which are indicated with same-numbered reference characters. The perforated polymer overcoat 162 defines a plurality of perforations 166. In some embodiments, the perforated polymer overcoat 162 covers the outer contour 58 and a portion of the elbow portion 52 that is proximate thereto. The filter structure 34d may also include a tail structure 168 that is made from a plurality of strands of the mesh 84 and extends from either end (or both ends) of the tubular sleeve 112. In some embodiments, the tail structure is held together with a crimp 170. The crimp 170 may be made with a radiopaque material.

An artifact of some filter assemblies 30 when in the implanted configuration 22 is a distortion of the sizes of the pores 76 at the elbow portion 52. Particularly, filter structures 34 that are arcuate about the second central axis 49 and define the outer contour 58 as arcuate about the lateral axis 54 are stretched or put in tension about the outer contour 58 of the elbow portion 52. The stretching causes the pores 76 on the outer contour 58 to increase in size, in some cases by as much as 70% or more. Accordingly, the porosity of the filter structure 34d sans the perforated polymer overcoat 162 is increased in the vicinity of the outer contour 58. The increased sizes of the pores 76 may diminish the filtering capability of the filter assembly 30. The elbow portion 52, being disposed upstream in the blood flow when deployed, is a particularly active filtering region of the filter assembly 30. In some embodiments, the perforated polymer overcoat 162 is loaded with or coated with an anti-thrombic compound.

Functionally, the perforations 166 enable the porosity of the filter structure 34d to be controlled so that the porosity in the region of the outer contour 58 is in substantial uniformity with the remainder of the filter structure 34d. In some embodiments, the perforations of the plurality of perforations are sized within a range of 0.2 to 0.8 millimeter diameter inclusive. Furthermore, the perforated polymer overcoat 162 may be of suitable flexibility to enable the filter assembly 30 to be straightened and collapsed for delivery. The tail structure 168 provides a snag for purposes of retrieving the filter assembly 30. The crimp 170, particularly when made of a radiopaque material, provides a location marker of the tail structure 168 for various imaging systems (e.g., x-rays). Loading the perforated polymer overcoat 162 polymer with the anti-thrombic compound can cause the overcoat 162 to elute the anti-thrombic compound over time, thereby reducing fibrin formation and preventing clots from forming and growing or otherwise preventing platelets from clumping and preventing clots from forming and growing on the perforated polymer overcoat 162.

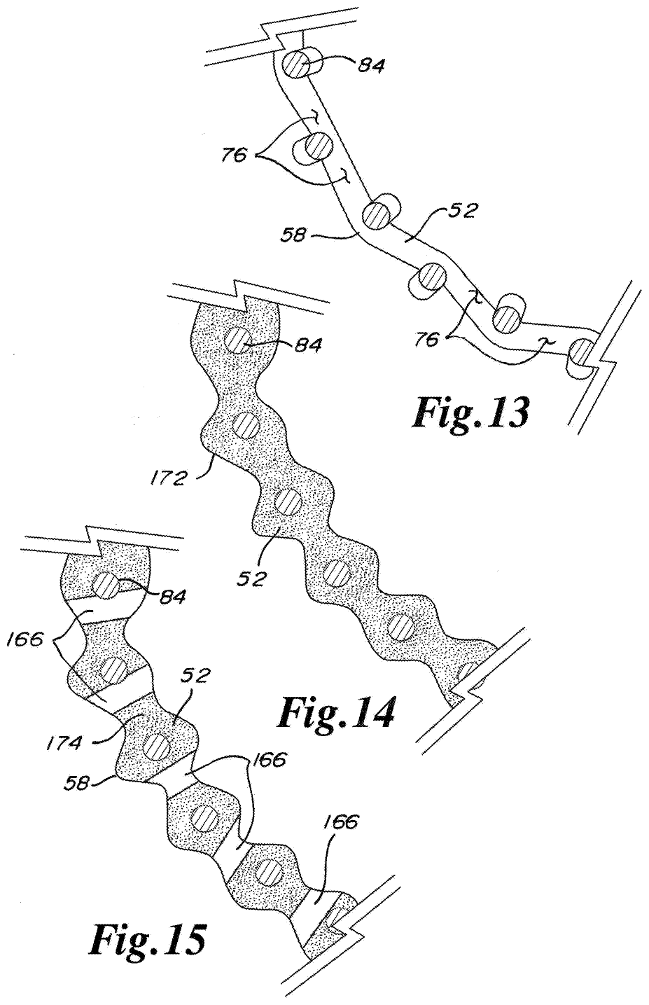

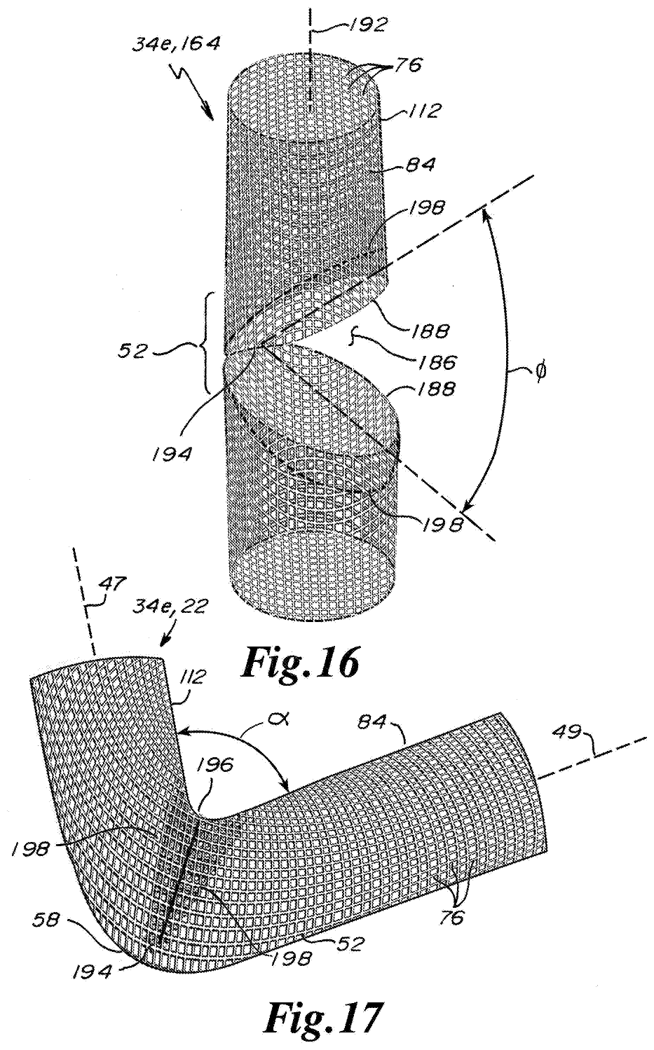

In fabrication, the tubular sleeve 112 the filter structure 34c may be braided about a substantially linear axis (FIG. 11) and fitted over a mandrel (not depicted) for shaping and heat setting, thereby defining the outer contour 58 of the elbow portion 52 (FIGS. 12 and 13). In some embodiments, a liquid polymer 172 may is applied over and in the region of the outer contour 58 of the elbow portion 52. The liquid polymer 172 may fill the pores 76 (FIG. 14) or at least partially fill the pores 76 of the mesh 84. The liquid polymer 172 sets to define a polymer coating 174, and the perforations 166 formed that pass through the thickness of the polymer coating 174 (FIG. 15).

The perforations 166 may be formed, for example, using a laser cutting process or a mechanical puncturing process. Various perforation techniques can be adapted to form the perforations 166 while not substantially compromising the structural integrity of the mesh 84. For example, laser cutting may utilize a laser intensity that is suitable for cutting the polymer coating 174 but that does not damage a mesh 84 that is metallic. In another example, a mechanical puncturing process may incorporate needles that come to a sharp point that deflects either the (metallic) mesh 84 or the needle punch upon incidence with the mesh 84.

The technique of applying the perforated polymer overcoat 162 is not limited to filter structures 34. Certain embodiments include support structures 32 that may perform a filtering function (e.g., the support structure 32f of FIG. 19), which may also be subject to distortion of the pores 76 in the implanted configuration 22. Accordingly, the perforated polymer overcoat 162 may find remedial application on support structures 32 as well.

While the embodiment of FIGS. 11 through 15 depict a wire braid or weave for the mesh 84, those of ordinary skill in the art, in view of this disclosure, will recognize that the same pore expansion phenomenon can occur with other mesh forms (e.g., expansion meshes) and can apply the same technique thereto.

Referring to FIGS. 16 and 17, a filter structure 34e is depicted in the pre-implant configuration 164 and the implanted configuration 22, respectively, according to an embodiment of the disclosure. The filter structure 34e may include some components and attributes as the filter structure 34d, some of which are indicated with same-numbered reference characters.

In the pre-implant configuration 164, the filter structure 34e is characterized by a partial discontinuity 186 at the elbow portion 52 that forms mitered edges 188. The mitered edges 188 define a miter angle ϕ when the tubular sleeve structure 112 is substantially concentric with a linear axis 192. A hinge portion 194 bridges one side of the discontinuity 186. In the implanted configuration 22, the discontinuity 186 is closed to form a miter 196, with the hinge portion 194 aligned along the outer contour 58 of the implanted configuration 22. The miter angle ϕ may be sized so that a resulting angle α about the miter 196 of the implanted configuration 22 is congruent with the desired minimum projected angle θ. In some embodiments, the form of the implanted configuration 22 of the filter structure 34e is maintained by the support structure 32, for example support structures 32a or 32b. Other support structures 32 disclosed throughout this disclosure may also be utilized with the filter structure 34e.

Fabrication of the filter structure 34e may include braiding or weaving the tubular sleeve structure 112 of the filter structure 34e about a substantially linear axis and partially severing the tubular sleeve structure 112, leaving the hinge portion 194 in place. Folds or rolls 198 may be formed at the edges of the partial sever by folding or rolling the severed edges back along the inside of the tubular sleeve structure 112 to define the mitered edges 188. The degree to which the severed edges are folded or rolled back defines the miter angle ϕ of the filter structure 34e when in the implanted configuration 22. The filter structure 34e may be formed to the shape of the implanted configuration 22 using a mandrel (not depicted) and heat setting the shape.

Functionally, the pores 76 of the filter structure 34e experience less distortion in the implanted configuration 22 than do the pores 76 of certain filter structures 32, such as filter structure 32d. As a result, in some embodiments, the porosity along the outer contour 58 of the filter structure 34e in the implanted configuration 22 is not substantially increased, and may not require remedial attention, such as the perforated polymer overcoat 162 (FIG. 12).

Referring to FIGS. 18 and 19, a filter assembly 30f including a support structure 32f and a filter structure 34f is depicted according to an embodiment of the disclosure. For this embodiment, the mesh 84 of the filter structure 34f is a woven wire mesh. The filter structure 34f extends from the anchor leg 46 over a portion of the filter leg 48, including over the elbow portion 52. The support structure includes a pair of rails 222 that are integral with and extend in a proximal direction 216 from the filter structure 34f. The rails 222 have a wider or thicker cross section than the strands of the mesh 84 of the filter structure 34f. In some embodiments, the rails 222 extend in a distal direction 218 into the filter structure 34f. The rails 222 each include a proximal end portion 224 and a distal end portion 226. The proximal end portions 224 that may be coupled together to form a loop 228. Herein, “proximal” and “distal” are relative terms that refer generally to the direction of blood flows, with proximal being generally upstream of the blood flow from distal. For the support structure 32f, two rails 222 are depicted, but additional rails are also contemplated.

The filter structure 34f extends from a distal end 232 to a proximal end 234, defining an assembled length 236. The filter structure 34f may define a channel-shaped portion 238 at the proximal end 234 and transition to a closed neck portion 244 at the distal end 232 that defines the first opening 36 of the filter assembly 30f. In some embodiments, the first opening 36 is bounded by a hoop structure 242 that is integral with the filter structure 34f. The rails 222 may extend to the hoop structure 242. The channel-shaped portion 238 may define a cross-section 246 in a plane that is orthogonal to the second central axis 49. The cross-section may define, for example, a U-shape (depicted) or a V-shape. The channel-shaped portion 238 includes opposed lateral edge portions 248 separated by a web portion 252. The filter structure 34f is coupled to the rails 222 at the opposed lateral edge portions 248 and at the closed neck portion 244.

The rails 222 extend at beyond the proximal end 234 of the filter structure 34f to define an inside length 254 of the loop 228. The inside length 254 is defined as the distance along the second central axis 49 from the proximal end 234 of the filter structure 34, 34f to an intersection of an inside surface of the loop 228 with the second central axis 49 when the filter assembly 30, 30f is in a pre-implant configuration (FIG. 18). In some embodiments, a ratio of the assembled length 236 to the inside length 254 when in the pre-implant configuration 164 is in a range of 5 to 1 inclusive. In some embodiments, a ratio of the inside length 254 to the assembled length 236 in the pre-implant configuration 164 is in a range of 1.25 to 2.5 inclusive. In some embodiments, a ratio of the inside length 254 to the length assembled 236 in the pre-implant configuration 164 is in a range of 1.4 to 2 inclusive.

In fabrication, the filter structure 34f may be interlaced with the rails during formation of the mesh 84 of the filter structure 34f In this way, the rails 222 are incorporated into mesh 84. The distal end portions 226 of the rails 222 may be woven into the mesh 84. In this way, the filter structure 34f captures the rails 222. The rails 222, being substantially stouter than the filter structure 34f, provide support for the filter structure 34f.

The distal end portions 226 of the rails 222 may be tightly woven into the mesh 84 at or proximate the closed neck portion 244. In contrast, the rails 222 may be loosely woven into the mesh 84 within the channel-shaped portion 238. Other forms of attaching the rails to the mesh 84 may also be implemented, for example tac welding. In some embodiments, the hoop structure 242 is formed by rolling or folding excess material of the mesh 84 at the first opening 36.

The rails 222 are coupled together at the proximal end portions 224 and formed to shape the loop 228. The loop 228 defines the inside length 254 beyond the proximal end 234 of the filter structure 34f when in the pre-implant configuration 164. The proximal end portions 224 of the rails 222 may be coupled, for example, with a crimp 258, which may comprise a radiopaque material. Other techniques for coupling the proximal end portions 224 of the rails 222 include, for example, twisting together, fusion, and tac welding. The filter assembly 30f is formed to shape (e.g., with mandrel) and heat set.

Functionally, the tight weave of the mesh 84 about the distal end portions 226 of the rails 222 may secure the rails 222 to the filter structure 34f. Conversely, the loose weave of the mesh 84 about the rails 222 within the channel-shaped portion 238 enables a sliding action between the rails 222 and the channel-shaped portion 238. The sliding fit of the loose weave in combination with the inside length 254 facilitates collapsing the filter assembly 30f for deployment. Herein, to “collapse” the filter assembly 30 refers to substantially straightening the filter assembly 30 about a linear axis and constricting the filter assembly 30 to within a reduced diameter (typically about two millimeters) for insertion into a delivery device.

When the mesh 84 is collapsed, the pores 76 are elongated, causing the major dimension 86 (FIG. 8) of mesh 84 to increase. The cumulative effect of the elongation of the pores 76 cause the collapsed mesh 84 to extend axially. Because of the loose weave between the rails 222 and the channel-shaped portion 238, the channel portion 238 slides over the rails 222 toward the proximal end portions 224. The inside length 254 provided by the support structure 32f provides space within the loop 228 for the cumulative elongation of the filter structure 34f. The sliding action also accommodates repositioning of the channel-shaped portion 238 relative to the rails 222 when transitioning between the pre-implant configuration 164 and the implanted configuration 22, which limits stresses imposed on the mesh 84.

The hoop structure 242 may enhance separation of the rails 222 from each other when transitioning between a collapsed configuration to the implanted configuration 22. The hoop structure 242 may also provide some biasing of the first opening 36 against the wall of the host artery. The loop 228 assures capture of the filter structure 34f within the support structure 32f. The radiopaque crimp 258, when utilized, serves as a location marker for various imaging systems. The crimp 258 can also function as a snare to facilitate retrieval of the filter assembly 30f. The larger cross section of the rails 222 relative to the strands of the mesh 84 enable the rails 222 to support and shape the filter structure 34f, causing the filter structure 34f to conform to the heat set shape of the rails 222. The cross sections of the rails 222 also provide spring biasing of the filter leg portion 48 for seating against the roof of the aortic arch 31.

In some embodiments, the rails 222 are made of wire. The wire is of substantially heavier gauge than that of the mesh 84. In some embodiments, the diameter of the wire forming the rails 222 is in a range of 100 to 500 micrometers inclusive; in some embodiments, in a range of 100 to 350 micrometers inclusive. In some embodiments, the cross-sections of the rails 222 may range from 0.03 to 0.4 square millimeters inclusive.

Referring to FIGS. 20 through 29, a filter assembly 30g including a support structure 32g and a filter structure 34g is depicted according to an embodiment of the disclosure. The filter assembly 30g includes some of the same components and attributes as the filter assembly 30f, some of which are indicated with same-numbered reference characters. The support structure 32g includes an anchor portion 302 distal to the rails 222. In some embodiments, the anchor portion 302 is an expanded tube structure 304. The rails 222 may be unitary with the expanded tube structure 304.

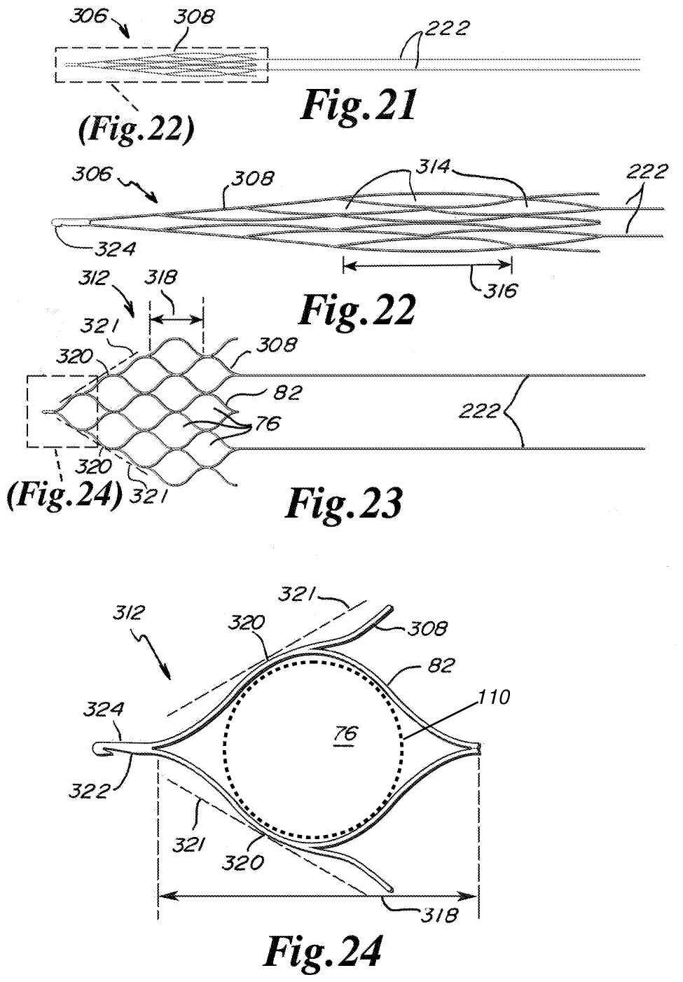

A planar projection 306 of a pre-expanded tube structure 308 prior to expansion is depicted at FIGS. 21 and 22. A planar projection 312 of the expanded tube structure 304 after expansion is depicted at FIGS. 23 and 24. Pre-expansion pores 314 define an elongate shape having a pre-expansion major dimension 316 (FIG. 22). After expansion, the pores 76 define a major dimension 318. Because of the lateral expansion of the pre-expanded tube structure 308, the post-expansion major dimension 318 is substantially shorter than the pre-expansion major dimension 316. The pores 76 define the coarse mesh 82 relative to the fine mesh 84 of the filter structure 34g. In some embodiments, a distal edge 320 of the anchor portion 302 defines a taper 321 at a distal extremity 322. The distal extremity 322 may define a hook portion 324 (FIG. 24). In some embodiments, the rails 222 are also formed from the pre-expanded tube structure 308 (FIG. 21), thereby being unitary with the anchor portion 302. In some embodiments, the pre-expanded tube structure 308 has a wall thickness that is substantially greater than the diameter of the strands of the mesh 84, and the tangential (height) dimension is tailored to provide the desired rigidity. In some embodiments, the wall thickness of the pre-expanded tube structure is within a range of 0.007 inches to 0.15 inches inclusive. The tangential dimension (height) of the rails 222 are cut to within a range of 0.007 inches to 0.04 inches inclusive. Accordingly, the rails 222 are substantially stouter than the mesh 84.

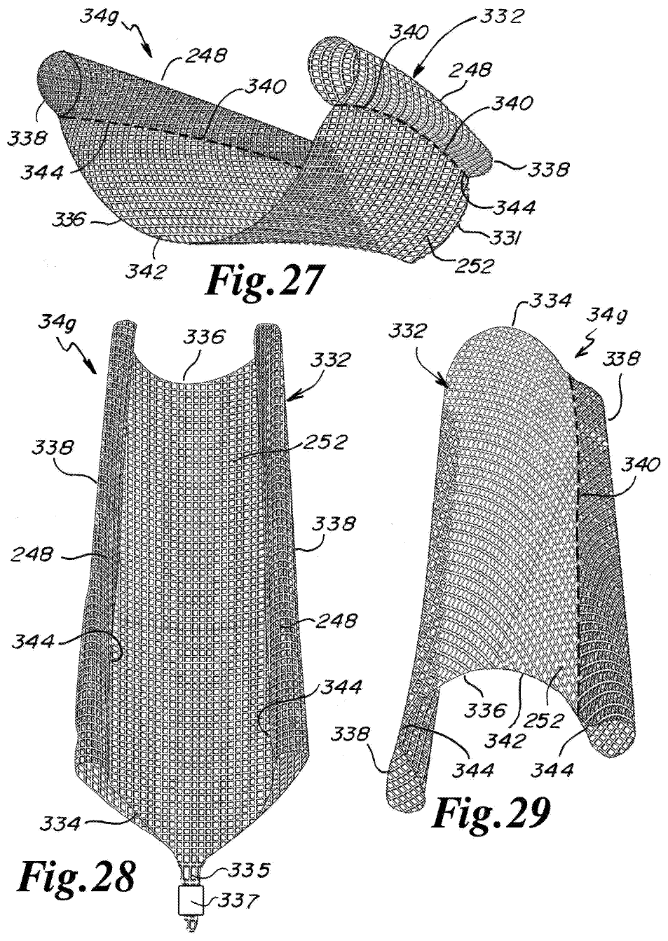

The filter structure 34g, depicted at FIGS. 27 through 29, may be a channel or “half pipe” structure 332, akin to the channel-shaped portion 238 of the filter structure 34f. The channel structure 332 includes a proximal end 334 and a distal end 336. A tail 335 may extend from the proximal edge 334 and include a crimp 337 of radiopaque material. The channel structure 332 may include hem structures 338 formed at the lateral edge portions 248. The rails 222 extend through the hem structures 338 to support the filter structure 34g. The rails 222 thereby capture the filter structure 34g between loop 228 and the anchor portion 302. Like the channel-shaped portion 238 of the filter structure 34f, the channel structure 332 may define the cross-section 246 having a U-shape or a V-shape.

In fabrication, the pre-expansion pores 314 and hook portion 324 are formed in the pre-expanded tube structure 308, for example in a laser cutting process. The pre-expanded tube structure 308 may also be trimmed to define the taper at the distal edge 320. In some embodiments, elongate segments 309 (depicted in phantom in FIGS. 25 and 26) of the pre-expanded tube structure 308 are also cut from the pre-expanded tube structure 308 to define the rails 222. The pre-expanded tube structure 308 is expanded to form the anchor portion 302.

For the filter structure 34g, the hem structures 338 may be formed by folding and fastening lateral extremities 344 of the mesh 84 to the web portion 252 to define the lateral edge portions 248. Fastening of lateral extremities 344 to the web portion 252 may be accomplished, for example, with an interwoven wire 340. The hem structures 338 are slid over the open rails 222 and brought into contact with the anchor portion 302. In some embodiments, the distal end 336 of the channel structure 332 is attached to the anchor portion 302, for example, with wire sutures or crimps 346. The crimps 346 may include a radiopaque material. The distal end 336 of the filter structure 34g may be disposed inside the second opening 38 the anchor portion 302, wrapped around the second opening 38 of anchor portion 304, or brought into abutment or otherwise align with the second opening 38 of the anchor portion 304. In the abutment option, the distal end 336 of the filter structure 34g defines a radius that is substantially the same as the radius of the anchor leg 46 about the first central axis 47, thereby reducing the effect of any axial discontinuity at the second opening 38.

The proximal end portions 224 of the rails 222 are formed to shape the loop 228 and define the inside length 254 beyond the proximal end 234 of the filter structure 34g. As with the filter assembly 30f, the proximal end portions 224 of the rails 222 may be coupled with a radiopaque crimp 258 (depicted), or by other techniques available to the artisan. The filter assembly 30g is formed to shape (e.g., with mandrel) and heat set.

In some embodiments, the filter structure 34g is fabricated from a polymer material. The pores 76 of the mesh 84 may be formed, for example, by laser cutting and/or expansion techniques. The hem structures 338 may be fabricated by fusing the lateral extremities 344 of the mesh 84 to the web portion 252.

Functionally, the hem structures 338 facilitate sliding of the filter structure 34g along the rails during the axial elongation that occurs when collapsing the filter assembly 30g for deployment. The hem structures 338 also facilitate assembly of the filter assembly 30g without interlacing the mesh 84 onto the support structure 32, and also enable use of different materials (e.g., polymer) for the filter structure 34g. As with the filter assembly 30f, the sliding action accommodates repositioning of the channel-shaped portion 238 relative to the rails 222 when transitioning between the pre-implant configuration 164 and the implanted configuration 22.

When implanted in the aortic arch 31, filter structures 34 that have a channel-shaped portion 238 (e.g., filter structures 34a, 34f, and 34g) suspend the mesh 84 of the web portion 252 away from the roof of the aortic arch 31 and the ostia of the arteries, which reduces the effect of pore blockage. Consider a configuration where the mesh structure is in apposition with the aortic roof at the perimeter of the ostia. Essentially, all of the blood flowing into the respective artery must pass through those pores which are bounded by the perimeter of the respective ostium. If some of the pores become blocked, the flow area into the artery is partially obstructed. Should such blockage become significant, blood flow into the artery may be substantially compromised.

By suspending the mesh 84 away from the ostia, the blood flow into a given artery is spread over a larger area of the filter structure 34. Spreading the blood flow over a larger area (i.e., over more pores of the filter structure 34) mitigates the effect of pore blockage. That is, if emboli entrained in the blood flow blocks a pore 76 of filter structure 34, the blockage affects a smaller fraction of the total number of pores 76 through which blood flows. Also, should several pores 76 become blocked such that the blockage of pores becomes significant, the blood flow will flow around the significant blockage by utilizing more pores adjacent to the blockage. The suspension of web portion 252 away from the roof of the aortic arch 31 also facilitates the self-cleaning aspect of the filter assembly 30. The separation enables cross flows to occur through the filter structure 34 during the cardiac cycle that may dislodge emboli from the pores of the mesh 34, with the emboli being returned to the aortic arch and away from the inlets of the arteries.

It is noted that the web portion 252 does not need to be arcuate for to have the beneficial effect described above. The web portion may extend linearly between the rails and provide the same effect, provided that the location of the seating of the rails 222 against the aortic arch 31 provides adequate separation between the mesh 84 and the ostia of the filtered arteries.

The crimps 346 secure the distal end 336 the filter structure 34g to the anchor portion 302, thereby maintaining coverage of the elbow portion 52 with the filter structure 34g. The crimps 346, when including a radiopaque material, serve as a location marker of the junction between the filter structure 34g and the anchor portion 302 that can be viewed with various imaging systems. The crimp 337 of the tail 335 can function as a snare for retrieval of the filter assembly 30g.

The loop 228 assures capture of the filter structure 34g within the support structure 32g. The crimp 258 not only provide closure of the loop 228, but can also facilitate retrieval of the filter assembly 30g. A radiopaque crimp 258, when utilized, serves as a location marker for various imaging systems. The increased cross section of the rails 222 relative to the strands of the mesh 84 enable the rails 222 to support the filter structure 34g and to cause the filter structure 34g to conform to the heat set shape of the rails 222. The cross sections of the rails 222 also provides spring biasing of the filter leg portion 48 for seating against the roof of the aortic arch 31.

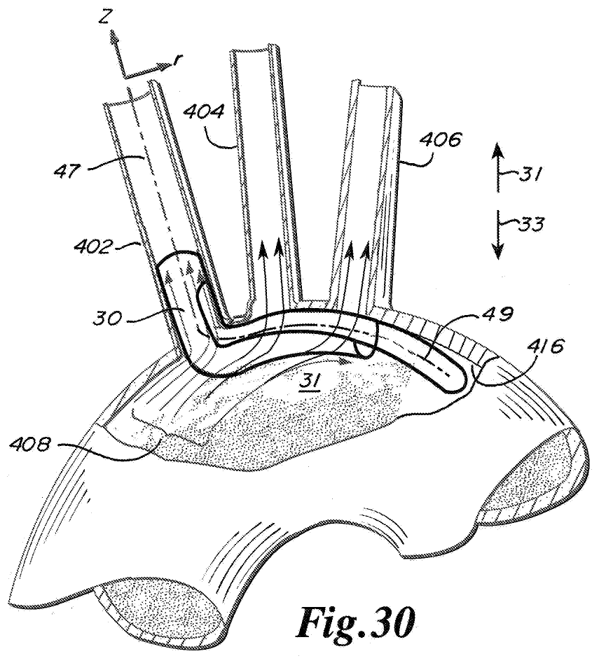

Referring to FIGS. 30 through 33, a three dimensional (3D) contoured shape 400 tailored to provide substantially continuous (sealed) posterior and anterior contact about the ostia of the heart is depicted according to an embodiment of the disclosure. The depiction of FIG. 30 portrays the filter assembly 30 implanted in the innominate artery 402 and extending over the ostia of the left carotid artery 404 and the left subclavian artery 406. A blood flow 408 is depicted as passing through the filter assembly 30 into the arteries 402, 404, and 406. Also depicted are the superior direction 33 and the inferior direction 35 of the filter assembly 30 when implanted, and the lateral projection plane 66. The lateral projection plane 66 is coplanar with first central axis 47 and centered about the second central axis 49. The projection plane 66 is defined as being “centered” about the second central axis 49, for example by a least-squares fit between the second central axis 49 and the projection plane 66.

In general and approximate terms, the lateral projection plane 66 is parallel to the coronal plane of the human body and orthogonal to an anterior direction 412 and posterior direction 414 of the human body. While depictions of the filter assembly 30 in FIGS. 30 through 33 correlate specifically with the features of the filter assembly 30f, the principles governing the 3D contoured shape 400 are similar for all filter assemblies 30 depicted herein, such that, in view of this disclosure, an artisan of ordinary skill can apply the governing principles mutatis mutandis for all filter assemblies 30 presented herein. The various arteries 402, 404, and 406 are not typically coplanar, and typically do not enter the aortic arch 31 normal to the aortic wall. Accordingly, to provide a snug fit between the filter assembly 30 and the aortic arch 31, it is advantageous to tailor the filter assembly 30 to conform to the aortic arch 31.

The depictions of FIGS. 31 through 33 present an outline 420 of the relevant aspects of the filter assembly 30, and include some of the same components and attributes presented throughout this disclosure, some of which are identified by same-numbered reference characters. These include the lateral edge portions 248, identified individually as a first lateral edge portion 248a and a second lateral edge portion 248b. Also included in the outline 420 is the first central axis 47, the second central axis 49, the anchor leg portion 46, the filter leg portion 48, the first opening 36, and the second opening 38. The second opening 38 is located at the junction between the anchor leg portion 46 and the filter leg portion 48.

A roof 416 of the aortic arch 31 presents an arcuate shape. As such, the lateral edges 248 of the filter 34 may also be arcuately shaped to better conform to the profile of the aortic roof 416. Because of the arcuate cross-section 246 of the web portion 252, the seating of the rails against the aortic roof 416 does not put the web portion 252 in apposition with the aortic roof 416. The benefit of such an arrangement is described attendant to filter assembly 30g at FIGS. 20 through 29. When implanted, the first central axis 47 in the depicted example is concentric with the innominate artery 402, and therefore generally is not normal to the aortic roof 416. Accordingly, as projected onto the lateral projection plane 66, the portion of the aortic roof 416 that contacts the first lateral edge portion 248a extends further in the superior direction 33 than does the portion of the aortic wall that contacts the second lateral edge portion 248b (FIG. 32). Ergo, the first lateral edge portion 248a may be configured to extend further in the superior direction 33 than is the second lateral edge portion 248b. The arcuate shape of the lateral edges 248 may also be characterized as arcing in the inferior direction 35.

The filter assembly 30 may also accommodate the non-linear arrangement of the ostia of the innominate artery 402, the left carotid artery 404, and the left subclavian artery 406. Generally, the left carotid artery 404 is located further in the anterior direction 412 than is the left subclavian artery 406. Accordingly, the lateral edge portions 248 may arc generally in the posterior direction 414 (FIG. 31), so that the second central axis 49 is in substantial alignment with the centers of the ostia of the arteries 404 and 406.

The filter assembly 30 may be configured during fabrication as outlined above and heat set to adopt the stated characteristics in the pre-implant configuration 164. Functionally, the conformance of the filter assembly 30 to the aortic wall prevents emboli from bypassing the filter structure 34 and entering the arteries 402, 404, and 406. The conformance also enables substantial contact along the aortic arch 31 without application of an excessive biasing. Any reduction in the biasing force generally reduces erosion and irritation of the aortic roof 416, and can also reduce distortions of the filter assembly 30.

Each of the disclosed embodiments defines a minimum projected angle θ, which may also be described in reference to the outline 420 of FIGS. 31 and 32. The minimum projected angle θ is a minimum angle defined by the first central axis 47 and a projection of a tangent line 422 that is tangent to the second central axis 49, as the line is projected onto the lateral projection plane 66. For the outline 420, a tangent point 424 is identified on the second central axis 49 where the tangent line 422 that defines the minimum (smallest) projected angle θ. Note that the trajectory of the tangent line 422 itself may not be parallel to the lateral projection plane 66, as depicted in FIG. 31. However, the depiction of FIG. 32 provides a view that is orthogonal to the lateral projection plane 66, and thus presents the projected angle θ.

The minimum projected angle θ of FIG. 32 defines a projected angle θ that is obtuse. However, acute projected angles θ angle are also contemplated and disclosed herein (e.g., at FIG. 4). In some embodiments, the projected angle θ is in a range of 40 degrees to 80 degrees inclusive. In some embodiments, the projected angle θ is in a range of 50 degrees to 70 degrees inclusive.

Each of the additional figures and methods disclosed herein can be used separately, or in conjunction with other features and methods, to provide improved devices and methods for making and using the same. Therefore, combinations of features and methods disclosed herein may not be necessary to practice the disclosure in its broadest sense and are instead disclosed merely to particularly describe representative and preferred embodiments.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to “embodiment(s)”, “disclosure”, “present disclosure”, “embodiment(s) of the disclosure”, “disclosed embodiment(s)”, and the like contained herein refer to the specification (text, including the claims, and figures) of this patent application that are not admitted prior art. Herein, references to “proximal” and associated derivative terms refer to a direction or position that is toward the surgeon or operator. References to “distal” and associated derivative terms refer to a direction or position that is away from the surgeon or operator.

For purposes of interpreting the claims, it is expressly intended that the provisions of 35 U.S.C. 112(f) are not to be invoked unless the specific terms “means for” or “step for” are recited in the respective claim.

What is claimed is:

1. A filter assembly for filtering blood entering an artery from an aortic arch, comprising:
a filter structure and a support structure that cooperate to define an anchor leg and a filter leg, said anchor leg defining a distal opening and extending along a first central axis, said first central axis extending in an inferior direction from said distal opening toward an elbow portion of said filter leg, said filter leg being proximal to said anchor leg and defining and extending along a second central axis, said filter leg including said elbow portion and an extension portion, said elbow portion extending from said anchor leg and separating said anchor leg from said extension portion,
wherein:
said support structure extends from said elbow portion into said extension portion; and
said filter structure is configured to slide on said support structure.

2. The filter assembly of claim 1, wherein said support structure includes at least one rail that extends into said extension portion, said filter structure being slidable on said rail.

3. The filter assembly of claim 2, wherein said at least one rail forms a loop proximal to said filter structure.

4. The filter assembly of claim 3, wherein said loop is configured as a snare portion for retrievability of said filter assembly.

5. The filter assembly of claim 4, comprising a crimp attached to a proximal end of said loop.

6. The filter assembly of claim 5, wherein said crimp includes a radiopaque material.

7. The filter assembly of claim 3, wherein:
said filter assembly defines an assembled length that extends from said first central axis at a distal opening of said anchor leg to an inside surface of said loop at said second central axis when said filter assembly is in a pre-implant configuration;
said loop defines an inside length along said second central axis that extends from a proximal end of said filter structure to said inside surface of said loop when said filter assembly is in a pre-implant configuration; and
a ratio of said assembled length to said inside length is in a range of 5 to 1 inclusive.

8. The filter assembly of claim 7, wherein said anchor leg includes a tail portion that extends distally beyond said assembled length, said tail portion being configured as a snare portion for retrievability of said filter assembly.

9. The filter assembly of claim 8, wherein said snare portion includes a hook structure.

10. The filter assembly of claim 8 wherein said tail portion includes a crimp attached to a distal end of said tail portion.

11. The filter assembly of claim 10, wherein said crimp includes a radiopaque material.

12. The filter assembly of any one of claims 1-11, wherein said filter structure and said support structure are interlaced.

13. The filter assembly of claim 12, wherein said filter structure is one of a braided structure and a woven structure

14. The filter assembly of claim 13, wherein:
said filter structure and said support structure are tightly interlaced at said elbow portion to secure said filter structure to said support structure; and
said filter structure and said support structure are loosely interlaced at said extension portion to enable said filter structure to slide over said support structure.

15. The filter assembly of any one of claims 1-11, wherein said filter structure includes opposed lateral edges that are lateral to said second central axis, each of said opposed lateral edges including a hem structure that enables said filter structure to slide over said support structure.

16. The filter assembly of any one of claims 1-11, wherein said extension portion of said filter structure defines an arcuate cross-section orthogonal to said second central axis that partially surrounds said second central axis, said arcuate cross section extending away from said central axis in said inferior direction.

17. The filter assembly of claim 16, wherein said arcuate cross-section defines one of a U-shape and a V-shape.

18. The filter assembly of claim 16, wherein a distal end of said filter structure is fastened to said anchor leg.

19. The filter assembly of claim 18, wherein said distal end of said filter structure defines a closed neck.

20. The filter assembly of claim 18, wherein said distal end of said filter structure is fastened to said anchor leg with crimps.

21. The filter assembly of claim 20, wherein said crimps include a radiopaque material suitable for visualization with an imaging system.

22. The filter assembly of claim 19, wherein said closed neck wraps around anchor leg.

23. The filter assembly of claim 19, wherein said distal end of said filter structure abuts against said anchor leg to define a diameter that is substantially the same as a diameter of said anchor leg.

24. The filter assembly of claim 23, wherein said anchor support structure defines a first area porosity that is within a range of 60% to 98% inclusive.

25. The filter assembly of claim 24, wherein a mesh used to fabricate said filter structure defines a second area porosity that is within a range of 50% to 98% inclusive.

26. The filter assembly of claim 1, wherein a nominal pore size defined by said support structure is greater than a nominal pore size defined by said filter leg.

27. The filter assembly of claim 26, wherein said nominal pore size of said support structure is within a range of 0.5 to 8 millimeters inclusive.

28. The filter assembly of claim 27, wherein said nominal pore size of said support structure is within a range of 0.5 to 5 millimeters inclusive.

29. The filter assembly of claim 28, wherein said nominal pore size of said support structure is within a range of 0.5 to 3 millimeters inclusive.

30. The filter assembly of claim 28, wherein said nominal pore size of said filter mesh of filter structure is within a range of 0.2 to 0.8 millimeter inclusive.

31. The filter assembly of claim 25, wherein a ratio of said nominal pore size of said support structure to said nominal size of said filter mesh of filter structure is within a range of 2.5 to 55 inclusive.

32. The filter assembly of claim 31, wherein a ratio of said nominal pore size of said support structure to said nominal size of said filter mesh of filter structure is within a range of 2.5 to 40 inclusive.

33. The filter assembly of claim 32, wherein a ratio of said nominal pore size of said support structure to said nominal size of said filter mesh of filter structure is within a range of 2.5 to 25 inclusive.

34. The filter assembly of any one of claim 1, wherein, when said filter assembly is in an implant configuration, a lateral projection of said first central axis and said second lateral axis defines a minimum projected angle, said minimum projected angle being within a range of 40 degrees to 80 degrees inclusive.

35. The filter assembly of claim 34, wherein said minimum projected angle is within a range of 50 degrees to 70 degrees inclusive.

36. A filter assembly for filtering blood entering an artery from an aortic arch, comprising:
an anchor leg and a filter leg that extends from said anchor leg, said filter leg including an elbow portion, said anchor leg defining a first central axis that extends in a first direction from said anchor leg away from said elbow portion;
a support structure extending from said anchor leg along said filter leg, said support structure including a pair of support rails that extend beyond said elbow portion along said filter leg;
a first support rail of said pair of support rails defining a first shape beyond said elbow portion; and
a second support rail of said pair of support rails defining a second shape beyond said elbow portion,
wherein said second shape extends further in said first direction than said first shape.

37. The filter assembly of claim 36, wherein said first shape and said second shape each arc toward a second direction, said second direction being opposite said first direction.

38. The filter assembly of claim 36, wherein said first shape and said second shape each arc toward a first lateral direction, said first lateral direction being perpendicular to said first direction.

39. The filter assembly of any one of claims 36-38, wherein said anchor leg is configured to anchor said filter assembly in a brachiocephalic artery.

40. The filter assembly of claim 39, wherein said first shape and said second shape are each configured for continuous contact along a roof of an aortic arch, the continuous contact of said first shape being anterior to the continuous contact of said second shape

41. The filter assembly of claim 36, comprising a filter structure coupled to said pair of rails, said filter structure including a web portion that extends between said pair of rails.

42. The filter assembly of claim 41, wherein filter structure defines an arcuate cross-section orthogonal to said second central axis, said arcuate cross section extending in a second direction that is opposite said first direction.

43. The filter assembly of claim 42, wherein said arcuate cross-section defines one of a U-shape and a V-shape.

44. A filter assembly for filtering blood entering an artery from an aortic arch, comprising:
a filter structure and a support structure that cooperate to define an anchor leg and a filter leg, said anchor leg defining a distal opening and extending along a first central axis, said first central axis extending in an inferior direction from said distal opening toward an elbow portion of said filter leg, said filter leg being proximal to said anchor leg and defining and extending along a second central axis, said filter leg including said elbow portion and an extension portion, said elbow portion extending from said anchor leg and separating said anchor leg from said extension portion,
wherein a perforated polymer coating covers an outer contour of said elbow portion.

45. The filter assembly of claim 44, wherein said perforated polymer coating covers said outer contour of said elbow portion and defines a plurality of perforations that pass through said perforated polymer coating.

46. The filter assembly of claim 45, wherein the perforations of said plurality of perforations are sized within a range of 0.2 to 0.8 millimeter diameter inclusive.

47. The filter assembly of claim 46, wherein said perforated polymer coating defines an area porosity that is within a range of 60% to 98% inclusive.

48. A method of making the filter assembly of any one of claims 44-47, comprising:
coating said outer contour of said elbow portion with a polymer; and
forming said plurality of perforations through said polymer.

49. The method of claim 48, wherein said polymer is applied as liquid and allowed to harden before the step of forming.

50. The method of claim 48, wherein said plurality of perforations are formed by a laser cutting process.

51. The method of claim 48, wherein said filter assembly is formed to shape over a mandrel and heat set to form said elbow portion prior to the step of coating.

52. A method of collapsing a filter assembly for vascular delivery, comprising:
bending a filter assembly from an implant configuration to a pre-implant configuration;
collapsing said filter assembly toward a central axis of said pre-implant configuration; and
sliding at least a portion of a filter structure of said filter assembly along a support structure of said filter assembly during the step of collapsing to elongate said filter structure along said central axis of said pre-implant configuration.

53. The method of claim 52, wherein said filter structure slides along a rail of said support structure during the step of sliding.

54. The method of claim 53, wherein said filter structure includes a hem that slides along said rail of said support structure during the step of sliding.

55. The method of any one of claims 52-54, wherein said filter assembly in the steps of bending and collapsing is made of super-elastic material.

56. The method of claim 55, wherein said superelastic material is one of a nickel titanium alloy and a cobalt-chromium-nickel-molybdenum-iron alloy.

57. A method of forming rails on a support structure of a filter assembly, comprising:
forming a plurality of pre-expansion pores at a first end portion of a tube to define a pre-expansion anchor portion;
cutting at least one segment proximal to said pre-expansion anchor portion to form at least one rail extending proximal to said pre-expansion anchor portion; and
expanding said tube to define an expanded anchor portion.

58. The method of claim 57, wherein a ratio of a length that said rail extends from said expanded anchor portion to a length of said expanded anchor portion is in range of 0.2 to 1.5 inclusive.

59. The method of claim 57, comprising forming a taper at a distal end of said pre-expansion anchor portion.

60. The method of claim 57, comprising:
coupling a filter structure to said at least one rail portion, said filter structure including a proximal end; and
closing said at least one rail portion to form a loop with said proximal end of said filter portion to support said filter structure.

61. The method of claim 60, wherein the step of coupling said filter structure includes capturing said at least one rail portion within a hem structure of said filter structure.

62. The method of claim 57, wherein said at least one rail portion in the step of cutting at least one segment is two rail portions.

63. The method of claim 62, wherein the step of closing includes joining proximal ends of said two rail portions together.

64. The method of claim 57, wherein said tube in the step of forming said plurality of pre-expansion pores is a circular tube.

65. The method of any one of claims 57-64, wherein the steps of cutting are performed with a laser.

66. A method of forming a filter assembly for a blood filter, comprising:
forming a tubular sleeve structure defining a substantially linear central axis, said tubular sleeve structure defining a wall porosity;
partially severing said tubular sleeve structure to form severed edges that are bridged by a hinge portion, said hinge portion extending along one side of said tubular sleeve structure;
rolling or folding said severed edges back along an inside of said tubular sleeve structure to define opposed mitered edges, said opposed mitered edges defining a miter angle when said tubular sleeve defines said substantially linear central axis; and
closing said miter angle about said hinge portion to define an elbow shape.

67. The method of claim 66, comprising mounting said tubular sleeve on a mandrel to close said miter angle.

68. The method of claim 67, comprising heat setting said tubular sleeve on said mandrel.

69. The method of claim of any one of claims 66-68, comprising sliding said sleeve structure over a support structure to close said miter angle.

70. A filter assembly for filtering blood flowing into an artery, comprising:
a tubular support structure that defines a first open end and a second open end that is opposed to said first open end, said tubular support structure having a tubular wall that defines a wall area porosity, said tubular support structure being curved to define a first leg portion and a second leg portion, said second leg portion including an elbow portion that extends from said first leg portion, said first leg portion defining said first open end and a first central axis, said second leg portion defining said second open end and a second central axis, said first central axis and said second central axis defining a minimum projected angle of said first central axis and said second central axis that is less than 180 degrees; and
a filter structure defining a filter area porosity and being coupled to said second leg portion of said tubular support structure,
wherein:
said filter assembly defines an inside portion that faces inward and an opposed outside portion that faces outward;
said filter structure and said tubular support structure define a combined area porosity that is less than said wall area porosity; and
said filter structure is arranged so that at least part of said outside portion of said filter assembly defines said combined area porosity at said elbow portion and said second leg portion, and at least part of said inside portion defines said wall area porosity at said second leg portion.

71. The filter assembly of claim 70, wherein said minimum projected angle is an obtuse angle.

72. The filter assembly of claim 70, wherein said minimum projected angle is an acute angle.

73. The filter assembly of claim 71, wherein said minimum projected angle is in a range of 40 degrees to 80 degrees inclusive.

74. The filter assembly of claim 73, wherein said minimum projected angle is in a range of 50 degrees to 70 degrees inclusive.

75. The filter assembly of claim 70, wherein said filter structure is disposed on an interior of said tubular support structure.

76. The filter assembly of claim 70, wherein said filter structure is disposed on an exterior of said tubular support structure.

77. The filter assembly of any one of claims 70-76, wherein said filter structure is attached to said tubular support structure with at least one of a threaded wire, a plurality of stitches, and a plurality of point-wise tack welds.

78. The filter assembly of claim 77, wherein said tubular support structure includes one of a braided structure and a woven structure, said tubular support structure including a plurality of pores defined therethrough.

79. The filter assembly of claim 77, wherein said tubular support structure is a coarse wire mesh.

80. The filter assembly of claim 79 wherein said coarse wire mesh includes wire having a diameter in a range of 100 micrometers to 300 micrometers inclusive and defining pore sizes in a range of three millimeters to five millimeters inclusive, said coarse wire mesh being one of a braided structure and a woven structure.

81. The filter assembly of claim 79, wherein said coarse wire mesh is formed from a single continuous wire.

82. The filter assembly of claim 81, wherein said continuous wire is composed of a material that includes one of a cobalt-chromium-nickel-molybdenum-iron alloy and a nickel-titanium alloy.

83. The filter assembly of claim 70, wherein said filter structure is a two-dimensional structure that conforms to a shape of said tubular support structure when coupled to said tubular support structure.

84. The filter assembly of claim 70, wherein said filter structure is one of a braided structure and a woven structure that is integrated with said elbow portion and said second leg portion.

85. The filter assembly of any one of claims 70-76, wherein said filter structure is a fine wire mesh.

86. The filter assembly of claim 85, wherein said fine wire mesh is braided or woven with wire having a diameter in a range of 30 micrometers to 100 micrometers inclusive.

87. The filter assembly of claim 86, wherein said fine wire mesh defines a plurality of non-circular pores, each having a nominal major dimension in a range of 200 micrometers to 800 micrometers inclusive.

88. The filter assembly of claim 87, wherein said fine wire mesh is woven or braided from a single wire.

89. The filter assembly of claim 88, wherein said single wire is of a super elastic material.

90. The filter assembly of claim 89, wherein said super elastic material is NITINOL.

91. A method of manufacturing the filter assembly, comprising:
forming said tubular support structure about a substantially linear axis;
fitting said tubular support structure over a curved mandrel to define said minimum projected angle;
heat treating said tubular support structure on said mandrel; and
coupling said filter structure to said elbow portion and said leg portion.

92. The method of claim 91, wherein said filter structure is a tubular sleeve structure defining a plurality of apertures formed on a first side thereof, said plurality of apertures being arranged so that said inside portion of said filter assembly defines said wall area porosity of said tubular support structure through said plurality of apertures.

93. The method of claim 92, wherein one or more of said plurality of apertures is arranged on said first side for substantial alignment with ostia of arteries that branch from an aortic arch when said filter assembly is implanted in said aortic arch.

94. A method of manufacturing the filter assembly of any one of claims 92-93, comprising:
forming said tubular sleeve structure of said filter structure about a substantially linear axis;
fitting said tubular sleeve structure over a mandrel, said mandrel including a plurality of apertures on one side that pass through a wall of said mandrel into a hollow defined by said mandrel;
heat treating said tubular support structure on said mandrel;
forming a plurality apertures in said tubular sleeve structure that pass through said plurality of apertures of said mandrel; and
coupling said filter structure to said elbow portion and said leg portion of said tubular support structure.

95. The method of claim 94, wherein the step of coupling said filter structure to said elbow portion and said second leg portion of said tubular support structure includes arranging said filter structure on an exterior of said tubular support structure.

96. The method of claim 91, wherein the step of forming said tubular support structure is one of a weaving or a braiding process.

97. A filter assembly, comprising:
a tubular support structure that defines a first open end and a second open end that is opposed to said first open end, said tubular support structure having a tubular wall that defines a wall area porosity, said tubular support structure being curved to define a first leg portion and a second leg portion separated by an elbow portion, said first leg portion defining said first open end and a first central axis, said second leg portion defining said second open end and a second central axis, said first central axis and said second central axis intersecting to define an apex angle that is less than 180 degrees, said apex angle defining a central plane of said tubular support structure; and
a filter structure defining a filter area porosity and being coupled to said elbow portion and said second leg portion of said tubular support structure,
wherein:
said filter assembly defines an inside portion that faces toward said apex angle and an opposed outside portion that faces away from said apex angle;
said filter structure and said tubular support structure define a combined area porosity that is less than said wall area porosity; and
said filter structure is arranged so that at least part of said outside portion of said filter assembly defines said combined area porosity at said elbow portion and said second leg portion, and at least part of said inside portion defines said wall area porosity at said second leg portion.
